(19) United States
(12) Patent Application Publication
(10) Pub. No.: US 2011/0269139 A1
(43) Pub. Date: Nov. 3, 2011

(54) BIOMARKERS AND METHODS FOR DETERMINING SENSITIVITY TO EPIDERMAL GROWTH FACTOR RECEPTOR MODULATORS

(75) Inventors: Ji Gao, Cranbury, NJ (US); Gayle M. Wittenberg, Plainsboro, NJ (US); Douglas Michael Robinson, Hanover, MA (US); Ashok Ramesh Dongre, Newtown, PA (US)

(73) Assignee: Bristol-Myers Squibb Company

(21) Appl. No.: 13/143,228

(22) PCT Filed: Jan. 6, 2010
(86) PCT No.: PCT/US10/20219

§ 371 Date: Jul. 5, 2011

Related U.S. Application Data
(60) Provisional application No. 61/142,721, filed on Jan. 6, 2009.

(51) Int. Cl. C12Q 1/68; C12Q 1/02; G01N 21/64; G01N 33/559; G01N 30/02; G01N 21/55; G01N 27/26; G01N 33/566; H01J 49/26

(52) U.S. Cl. 435/6.12; 435/7.21; 435/7.1; 435/7.92; 435/29; 436/501; 250/282; 73/61.52; 356/445; 204/450; 204/456

(57) ABSTRACT

The present invention provides methods useful for predicting the likelihood that a mammal that will respond therapeutically to a method of treating cancer comprising administering an EGFR modulator, and diagnostic methods and kits thereof.

This application claims benefit to provisional application U.S. Ser. No. 61/142,721 filed Jan. 6, 2009; under 35 U.S.C. §119(e). The entire teachings of the referenced application are incorporated herein by reference.

### FIELD OF THE INVENTION

The present invention relates generally to the field of pharmacogenomics, and more specifically to methods and procedures to determine drug sensitivity in patients to allow the identification of individualized genetic profiles which will aid in treating diseases and disorders.

### BACKGROUND OF THE INVENTION

Cancer is a disease with extensive histoclinical heterogeneity. Although conventional histological and clinical features have been correlated to prognosis, the same apparent prognostic type of tumors varies widely in its responsiveness to therapy and consequent survival of the patient.

Colorectal cancer remains the second leading cause of cancer deaths in the US and Europe. Despite advances in treatment options, for patients diagnosed with metastatic colorectal cancer the 5-year survival rate is a mere 10%. The challenge of successfully treating colorectal cancer at this late stage is in large part due to the heterogeneity of the disease. From person to person there is variability in the set of mutations that lead to the cancer. Within an individual there is variability in the mutant phenotype of primary and metastatic tumors. And even within a tumor there may be variability in the set of mutations present from one cell to the next.

Cetuximab is a chimeric monoclonal antibody which binds to the extracellular domain of the Epidermal Growth Factor Receptor (EGFR), preventing ligand binding and receptor activation. The activated EGFR turns on signaling pathways that are typically deregulated in cancer cells. EGFRs are frequently upregulated in colorectal cancer cells. For patients with metastatic colorectal cancer that has not responded to chemotherapy alone, Cetuximab increases both overall and progression free survival when compared with supportive care alone. However this enhancement comes from a subset of patients who respond to Cetuximab therapy.

New prognostic and predictive markers, which would facilitate an individualization of therapy for each patient, are needed to accurately predict patient response to treatments, such as small molecule or biological molecule drugs, in the clinic. The problem may be solved by the identification of new parameters that could better predict the patient's sensitivity to treatment. The classification of patient samples is a crucial aspect of cancer diagnosis and treatment. The association of a patient's response to a treatment with molecular and genetic markers can open up new opportunities for treatment development in non-responding patients, or distinguish a treatment's indication among other treatment choices because of higher confidence in the efficacy. Further, the pre-selection of patients who are likely to respond well to a medicine, drug, or combination therapy may reduce the number of patients needed in a clinical study or accelerate the time needed to complete a clinical development program (Cockett et al., Current Opinion in Biotechnology, 11:602-609 (2000)).

The ability to predict drug sensitivity in patients is particularly challenging because drug responses reflect not only properties intrinsic to the target cells, but also a host's metabolic properties. Efforts to use genetic information to predict drug sensitivity have primarily focused on individual genes that have broad effects, such as the multidrug resistance genes, mdr1 and mrp1 (Sonneveld, J. Intern. Med., 247:521-534 (2000)).

The search for biomarkers predictive of a therapeutic response to Cetuximab therapy has focused primarily on gene expression analysis of biopsied tumors and genomic analysis of biopsied tumor tissue. Working directly with tumor tissue can be beneficial: the information obtained comes directly from the tissue one is hoping to treat. However this comes with limitations. First, by the time cancer has metastasized, the genomic makeup of the secondary tumors may differ from the biopsied site or sites. Second, biopsies are invasive. As the disease progresses and new mutations accumulate, the biomarker status of a patient may change. It will not be practical to re-biopsy multiple tumors with a patient over the course of treatment.

The epidermal growth factor receptor (EGFR) and its downstream signaling effectors, notably members of the Ras/Raf/MAP kinase pathway, play an important role in both normal and malignant epithelial cell biology (Normanno et al., Gene, 366:2-16 (2006)) and have therefore become established targets for therapeutic development. Whereas the anti-EGFR antibody cetuximab and the EGFR small molecular tyrosine kinase inhibitors (TKIs) gefitinib and erlotinib have demonstrated activity in a subset of patients (Baselga et al., J. Clin. Oncol., 23:2445-2459 (2005)), their initial clinical development has not benefited from an accompanying strategy for identifying the patient populations that would most likely derive benefit. The hypothesis that only a relatively small number of tumors are “EGFR-pathway dependent” and therefore likely to respond to EGFR inhibitors might explain the limited clinical activity that is observed with this class of therapeutics. For example, in patients with refractory metastatic colorectal cancer clinical response rates with cetuximab consistently range from 11% in a monotherapy setting to 23% in a combination setting with chemotherapy (Cunningham et al., N. Engl. J. Med., 351:337-345 (2004)). To date, significant efforts have been focused on elucidating the mechanisms of sensitivity or resistance to EGFR inhibition, particularly through evaluation of EGFR protein expression, kinase domain mutations, and gene copy number.

While relative protein expression of the EGFR as measured by immunohistochemistry (IHC) has been demonstrated in many solid tumors (Ciardiello et al., Eur. J. Cancer, 39:1348-1354 (2003)), no consistent association between EGFR expression and response to EGFR inhibitors has been established. Clinical studies of cetuximab in a monotherapy setting and in combination with irinotecan in patients with mCRC failed to reveal an association between radiographic response and EGFR protein expression as measured by IHC (Cunningham et al., N. Engl. J. Med., 351:337-345 (2004); Saltz et al., J. Clin. Oncol., 22:1201-1208 (2004)). Furthermore, clinical responses have been demonstrated in patients with undetectable EGFR protein expression (Chung et al., J. Clin. Oncol., 23:1803-1810 (2005); Lenz et al., “Activity of cetuximab in patients with colorectal cancer refractory to both irinotecan and oxaliplatin”, Paper presented at: 2004 ASCO Annual Meeting Proceedings; Saltz, Clin. Colorectal Cancer, 5 Suppl. 2, S98-S100 (2005)). In comparison, clinical studies of erlotinib in NSCLC patients and gefitinib in ovarian cancer did demonstrate an association between EGFR expression, response, and survival (Schilder et al., Clin. Cancer Res., 11:5539-5548 (2005); Tsao et al., N. Engl. J. Med., 353:133-144 (2005)). The presence of somatic mutations in the tyrosine kinase domain, particularly in NSCLC has been extensively described (Janne et al., J. Clin. Oncol., 23:3227-3234 (2005)). In both preclinical and clinical settings, these mutations are found to correlate with sensitivity to gefitinib and erlotinib but not to cetuximab (Janne et al., J. Clin. Oncol., 23:3227-3234 (2005); Tsuchihashi et al., N. Engl. J. Med., 353:208-209 (2005)). In addition, the lack of EGFR kinase domain mutations in CRC patients suggests that such mutations do not underlie the response to cetuximab. EGFR gene copy number has also been evaluated as a potential predictor of response to EGFR inhibitors. Clinical studies of gefitinib demonstrated an association between increased EGFR copy number, mutational status, and clinical response (Cappuzzo et al., J. Natl. Cancer Inst., 97:643-655 (2005)). A similar association was identified in a small number of patients treated with the anti-EGFR monoclonal antibodies cetuximab and panitumumab (Moroni et al., Lancet Oncol., 6:279-286 (2005)). Additional potential predictive biomarkers have also been evaluated. For example, in glioblastoma patients, a significant association between co-expression of EGFRvIII and PTEN and response to EGFR small molecule inhibitors was found (Mellinghoff et al., N. Engl. J. Med., 353:2012-2024 (2005)).

The anti-tumor activity of cetuximab has been attributed to its ability to block EGFR ligand binding and ligand-dependent EGFR activation. Clinical activity of cetuximab has been shown in multiple epithelial tumor types (Bonner et al., N. Engl. J. Med., 354:567-578 (2006); Cunningham et al., N. Engl. J. Med., 351:337-345 (2004)), however responses continue to be seen in only a fraction of patients. Previous attempts to identify predictors of sensitivity or resistance as described above have focused on specific biomarkers rather than using genomic discovery approaches. In addition, RNA-, DNA- and protein-based markers have rarely been examined in the same patient population in a single study, making comparisons challenging. Non-plasma based biomarkers useful for determining sensitivity to EGFR modulators have been described in published PCT applications WO 2004/063709, WO 2005/067667, and WO 2005/094332.

Needed are new and alternative methods and procedures to determine drug sensitivity in patients, particularly those measurable in plasma, to allow the development of individualized genetic profiles which are necessary to treat diseases and disorders based on patient response at a molecular level.

### SUMMARY OF THE INVENTION

The invention provides methods and procedures for determining patient sensitivity to one or more Epidermal Growth Factor Receptor (EGFR) modulators. The invention also provides methods of determining or predicting whether an individual requiring therapy for a disease state such as cancer will or will not respond to treatment, prior to administration of the treatment, wherein the treatment comprises administration of one or more EGFR modulators. The one or more EGFR modulators are compounds that can be selected from, for example, one or more EGFR-specific ligands, one or more small molecule EGFR inhibitors, or one or more EGFR binding monoclonal antibodies.

In one aspect, the invention provides a method for predicting the likelihood a mammal will respond therapeutically to an EGFR modulator comprising the step of measuring the level of at least one biomarker in a biological sample of said mammal selected from the group consisting of: Fibronectin; histidine-rich glycoprotein; alpha2-HS glycoprotein; Complement component 3; and/or ras suppressor protein 1 in plasma; wherein an increase in the level of the at least one biomarker relative to a standard level indicates an increased likelihood the mammal will respond therapeutically to said EGFR modulator in treating cancer or other proliferative condition.

In one aspect, the invention provides a method for predicting the likelihood a mammal will respond therapeutically to a method of treating cancer comprising administering an EGFR modulator, wherein the method comprises: (a) measuring in the mammal the level of at least one biomarker selected from Fibronectin; histidine-rich glycoprotein; alpha2-HS glycoprotein; Complement component 3; and/or ras suppressor protein 1; (b) administering an EGFR modulator to said mammal; and (c) following the exposing of step (b), measuring in the biological sample the level of the at least one biomarker, wherein an increase in the level of the at least one biomarker measured in step (c) compared to the level of the at least one biomarker measured in step (a) indicates an increased likelihood that the mammal will respond therapeutically to the method of treating cancer. In one aspect, the at least one biomarker comprises Fibronectin; histidine-rich glycoprotein; and/or alpha2-HS glycoprotein. In yet another aspect, the at least one biomarker further comprises at least one additional biomarker selected from Table 2. In another aspect, the biological sample is a tissue sample comprising cancer cells and the method further comprises the step of determining whether the cancer cells have the presence of a mutated K-RAS, wherein detection of a mutated K-RAS indicates a decreased likelihood that that the mammal will respond therapeutically to the method of treating cancer.

The biological sample can be, for example, a tissue sample comprising cancer cells and the tissue is fixed, paraffin-embedded, fresh, or frozen.

In another aspect, the EGFR modulator is cetuximab and the cancer is colorectal cancer.

In another aspect, the invention is a method for predicting the likelihood a mammal will respond therapeutically to a method of treating cancer comprising administering an EGFR modulator, wherein the method comprises: (a) measuring in the mammal the level of at least one biomarker that comprises Fibronectin; (b) administering an EGFR modulator to said mammal; and (c) following the exposing of step (b), measuring in the biological sample the level of the at least one biomarker, wherein an increase in the level of the at least one biomarker measured in step (c) compared to the level of the at least one biomarker measured in step (a) indicates a decreased likelihood that the mammal will respond therapeutically to the method of treating cancer. In another aspect, the at least one biomarker further comprises at least one additional biomarker selected from Table 2. In another aspect, the method further comprises the step of determining whether the cancer cells have the presence of a mutated K-RAS, wherein detection of a mutated K-RAS indicates a decreased likelihood that that the mammal will respond therapeutically to the method of treating cancer.

In another aspect, the invention is a method for predicting the likelihood a mammal will respond therapeutically to a method of treating cancer comprising administering an EGFR modulator, wherein the method comprises: (a) measuring in the mammal the level of at least one biomarker that comprises histidine-rich glycoprotein; (b) administering an EGFR modulator to said mammal; and (c) following the exposing of step (b), measuring in the biological sample the level of the at least one biomarker, wherein an increase in the level of the at least one biomarker measured in step (c) compared to the level of the at least one biomarker measured in step (a) indicates an increased likelihood that the mammal will respond therapeutically to the method of treating cancer. In another aspect, the at least one biomarker further comprises at least one additional biomarker selected from Table 2. In another aspect, the method further comprises the step of determining whether the cancer cells have the presence of a mutated K-RAS, wherein detection of a mutated K-RAS indicates a decreased likelihood that that the mammal will respond therapeutically to the method of treating cancer.

In another aspect, the invention is a method for predicting the likelihood a mammal will respond therapeutically to a method of treating cancer comprising administering an EGFR modulator, wherein the method comprises: (a) measuring in the mammal the level of at least one biomarker that comprises alpha2-HS glycoprotein; (b) administering an EGFR modulator to said mammal; and (c) following the exposing of step (b), measuring in the biological sample the level of the at least one biomarker, wherein an increase in the level of the at least one biomarker measured in step (c) compared to the level of the at least one biomarker measured in step (a) indicates an increased likelihood that the mammal will respond therapeutically to the method of treating cancer. In another aspect, the at least one biomarker further comprises at least one additional biomarker selected from Table 2. In another aspect, the method further comprises the step of determining whether the cancer cells have the presence of a mutated K-RAS, wherein detection of a mutated K-RAS indicates a decreased likelihood that that the mammal will respond therapeutically to the method of treating cancer.

In another aspect, the invention is a method for predicting the likelihood a mammal will respond therapeutically to a method of treating cancer comprising administering an EGFR modulator, wherein the method comprises: (a) measuring in the mammal the level of at least one biomarker that comprises Complement component 3; (b) administering an EGFR modulator to said mammal; and (c) following the exposing of step (b), measuring in the biological sample the level of the at least one biomarker, wherein an increase in the level of the at least one biomarker measured in step (c) compared to the level of the at least one biomarker measured in step (a) indicates an increased likelihood that the mammal will respond therapeutically to the method of treating cancer. In another aspect, the at least one biomarker further comprises at least one additional biomarker selected from Table 2. In another aspect, the method further comprises the step of determining whether the cancer cells have the presence of a mutated K-RAS, wherein detection of a mutated K-RAS indicates a decreased likelihood that that the mammal will respond therapeutically to the method of treating cancer.

In another aspect, the invention is a method for predicting the likelihood a mammal will respond therapeutically to a method of treating cancer comprising administering an EGFR modulator, wherein the method comprises: (a) measuring in the mammal the level of at least one biomarker that comprises ras suppressor protein 1; (b) administering an EGFR modulator to said mammal; and (c) following the exposing of step (b), measuring in the biological sample the level of the at least one biomarker, wherein an increase in the level of the at least one biomarker measured in step (c) compared to the level of the at least one biomarker measured in step (a) indicates an increased likelihood that the mammal will respond therapeutically to the method of treating cancer. In another aspect, the at least one biomarker further comprises at least one additional biomarker selected from Table 2. In another aspect, the method further comprises the step of determining whether the cancer cells have the presence of a mutated K-RAS, wherein detection of a mutated K-RAS indicates a decreased likelihood that that the mammal will respond therapeutically to the method of treating cancer.

In another respect, the present invention is directed to a method for predicting the likelihood a mammal will respond therapeutically to an EGFR modulator comprising the step of measuring the level of at least one biomarker in a biological sample of said mammal selected from the group consisting of: Fibronectin in plasma; wherein a decrease in the level of the at least one biomarker relative to a standard level indicates an increased likelihood the mammal will respond therapeutically to said EGFR modulator in treating cancer or other proliferative condition, and vice versa.

In another respect, the present invention is directed to a method for predicting the likelihood a mammal will respond therapeutically to an EGFR modulator comprising the step of measuring the level of at least one biomarker in a biological sample of said mammal selected from the group consisting of: histidine-rich glycoprotein in plasma; wherein an increase in the level of the at least one biomarker relative to a standard level indicates an increased likelihood the mammal will respond therapeutically to said EGFR modulator in treating cancer or other proliferative condition, and vice versa.

In another respect, the present invention is directed to a method for predicting the likelihood a mammal will respond therapeutically to an EGFR modulator comprising the step of measuring the level of at least one biomarker in a biological sample of said mammal selected from the group consisting of: alpha2-HS glycoprotein in plasma; wherein an increase in the level of the at least one biomarker relative to a standard level indicates an increased likelihood the mammal will respond therapeutically to said EGFR modulator in treating cancer or other proliferative condition, and vice versa.

In another respect, the present invention is directed to a method for predicting the likelihood a mammal will respond therapeutically to an EGFR modulator comprising the step of measuring the level of at least one biomarker in a biological sample of said mammal selected from the group consisting of: Complement component 3 in plasma; wherein an increase in the level of the at least one biomarker relative to a standard level indicates an increased likelihood the mammal will respond therapeutically to said EGFR modulator in treating cancer or other proliferative condition, and vice versa.

In another respect, the present invention is directed to a method for predicting the likelihood a mammal will respond therapeutically to an EGFR modulator comprising the step of measuring the level of at least one biomarker in a biological sample of said mammal selected from the group consisting of: ras suppressor protein 1 in plasma; wherein an increase in the level of the at least one biomarker relative to a standard level indicates an increased likelihood the mammal will respond therapeutically to said EGFR modulator in treating cancer or other proliferative condition, and vice versa.

In certain circumstances, the presence of an activating K-RAS mutation may off-set, or decrease, the likelihood a mammal will respond therapeutically to an EGFR modulator. In some instances, such a decrease may be modest, in other circumstances, the decrease may be significant, depending upon the expression profile of the biomarkers of the present invention in said mammal, in addition to any other characteristics of the patient. In one aspect, overexpression of histidine-rich glycoprotein; alpha2-HS glycoprotein; Complement component 3; and/or ras suppressor protein 1, in addition to the presence of a K-RAS mutation may suggest the mammal will have a favorable response, an acceptable response, a decreased response, or a less than desirable response to an EGFR inhibitor depending upon the patients characteristics, though on balance may be expected to have a more favorable response to such an inhibitor relative to a mammal that had decreased levels of expression of these markers or no expression of these markers. In one aspect, decreased expression of fibronectin, in addition to the presence of a K-RAS mutation may suggest the mammal will have a favorable response, an acceptable response, a decreased response, or a less than desirable response to an EGFR inhibitor depending upon the patients characteristics, though on balance may be expected to have a more favorable response to such an inhibitor relative to a mammal that had increased level of expression of fibronectin or overexpression of fibronectin.

As used herein, respond therapeutically refers to the alleviation or abrogation of the cancer. This means that the life expectancy of an individual affected with the cancer will be increased or that one or more of the symptoms of the cancer will be reduced or ameliorated. The term encompasses a reduction in cancerous cell growth or tumor volume. Whether a mammal responds therapeutically can be measured by many methods well known in the art, such as PET imaging.

The mammal can be, for example, a human, rat, mouse, dog, rabbit, pig sheep, cow, horse, cat, primate, or monkey.

The method of the invention can be, for example, an in vitro method wherein the step of measuring in the mammal the level of at least one biomarker comprises taking a biological sample from the mammal and then measuring the level of the biomarker(s) in the biological sample. The biological sample can comprise, for example, at least one of serum, whole fresh blood, peripheral blood mononuclear cells, frozen whole blood, fresh plasma, frozen plasma, urine, saliva, skin, hair follicle, bone marrow, or tumor tissue.

The level of the at least one biomarker can be, for example, the level of protein and/or mRNA transcript of the biomarker. The level of the biomarker can be determined, for example, by RT-PCR or another PCR-based method, immunohistochemistry, proteomics techniques, or any other methods known in the art, or their combination.

In another aspect, the invention provides a method for identifying a mammal that will respond therapeutically to a method of treating cancer comprising administering of an EGFR modulator, wherein the method comprises: (a) measuring in the mammal the level of at least one biomarker selected from the biomarkers of Table 2; (b) administering an EGFR modulator to said mammal; and (c) following the exposing in step (b), measuring in said biological sample the level of the at least one biomarker, wherein a difference in the level of the at least one biomarker measured in step (c) compared to the level of the at least one biomarker measured in step (a) indicates that the mammal will respond therapeutically to the said method of treating cancer.

In another aspect, the invention provides a method for identifying a mammal that will respond therapeutically to a method of treating cancer comprising administering an EGFR modulator, wherein the method comprises: (a) administering an EGFR modulator to said mammal; and (b) following the exposing of step (a), measuring in said biological sample the level of at least one biomarker selected from the biomarkers of Table 2, wherein a difference in the level of the at least one biomarker measured in step (b), compared to the level of the at least one biomarker in a mammal that has not been exposed to said EGFR modulator, indicates that the mammal will respond therapeutically to said method of treating cancer.

In yet another aspect, the invention provides a method for testing or predicting whether a mammal will respond therapeutically to a method of treating cancer comprising administering an EGFR modulator, wherein the method comprises: (a) measuring in the mammal the level of at least one biomarker selected from the biomarkers of Table 2; (b) administering an EGFR modulator to said mammal; and (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

In another aspect, the invention provides a method for determining whether a compound inhibits EGFR activity in a mammal, comprising: (a) exposing the mammal to the compound; and (b) following the exposing of step (a), measuring in the mammal the level of at least one biomarker selected from the biomarkers of Table 2, wherein a difference in the level of said biomarker measured in step (b), compared to the level of the biomarker in a mammal that has not been exposed to said compound, indicates that the compound inhibits EGFR activity in the mammal.

In yet another aspect, the invention provides a method for determining whether a mammal has been exposed to a compound that inhibits EGFR activity, comprising: (a) exposing the mammal to the compound; and (b) following the exposing of step (a), measuring in the mammal the level of at least one biomarker selected from the biomarkers of Table 2, wherein a difference in the level of said biomarker measured in step (b), compared to the level of the biomarker in a mammal that has not been exposed to said compound, indicates that the mammal has been exposed to a compound that inhibits EGFR activity.

In another aspect, the invention provides a method for determining whether a mammal is responding to a compound that inhibits EGFR activity, comprising: (a) exposing the mammal to the compound; and (b) following the exposing of step (a), measuring in the mammal the level of at least one biomarker selected from the biomarkers of Table 2, wherein a difference in the level of the at least one biomarker measured in step (b), compared to the level of the at least one biomarker in a mammal that has not been exposed to said compound, indicates that the mammal is responding to the compound that inhibits EGFR activity.

As used herein, “responding” encompasses responding by way of a biological and cellular response, as well as a clinical response (such as improved symptoms, a therapeutic effect, or an adverse event), in a mammal.

The invention also provides an isolated biomarker selected from the biomarkers of Table 2. The biomarkers of the invention comprise sequences selected from the nucleotide and amino acid sequences provided in Table 2 and the Sequence Listing, as well as fragments and variants thereof

The invention also provides a biomarker set comprising two or more biomarkers selected from the biomarkers of Table 2.

The invention also provides kits for determining or predicting whether a patient would be susceptible or resistant to a treatment that comprises one or more EGFR modulators. The patient may have a cancer or tumor such as, for example, colorectal cancer, NSCLC, or head and neck cancer.

In one aspect, the kit comprises a suitable container that comprises one or more specialized microarrays of the invention, one or more EGFR modulators for use in testing cells from patient tissue specimens or patient samples, and instructions for use. The kit may further comprise reagents or materials for monitoring the expression of a biomarker set at the level of mRNA or protein.

In another aspect, the invention provides a kit comprising two or more biomarkers selected from the biomarkers of Table 2.

In yet another aspect, the invention provides a kit comprising at least one of an antibody and a nucleic acid for detecting the presence of at least one of the biomarkers selected from the biomarkers of Table 2. In one aspect, the kit further comprises instructions for determining whether or not a mammal will respond therapeutically to a method of treating cancer comprising administering a compound that inhibits EGFR activity. In another aspect, the instructions comprise the steps of: (a) measuring in the mammal the level of at least one biomarker selected from the biomarkers of Table 2; (b) exposing the mammal to the compound; and (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

The invention also provides screening assays for determining if a patient will be susceptible or resistant to treatment with one or more EGFR modulators.

The invention also provides a method of monitoring the treatment of a patient having a disease, wherein said disease is treated by a method comprising administering one or more EGFR modulators.

The invention also provides individualized genetic profiles which are necessary to treat diseases and disorders based on patient response at a molecular level.

The invention also provides specialized microarrays, e.g., oligonucleotide microarrays or cDNA microarrays, comprising one or more biomarkers having expression profiles that correlate with either sensitivity or resistance to one or more EGFR modulators.

The invention also provides antibodies, including polyclonal or monoclonal, directed against one or more biomarkers of the invention.

The invention will be better understood upon a reading of the detailed description of the invention when considered in connection with the accompanying figures.

### BRIEF DESCRIPTION OF THE FIGURES

FIG. 1 provides LCMS profiling data showing that fibronectin is present in higher concentrations in the plasma of Non-Responders with an AUC of 0.78, while HPRG and AHSG are both present at higher concentrations in the Responder population, with AUCs of 0.77 and 0.76, respectively.

FIG. 2 provides an ROC analysis and Log Rank Test for the FN biomarker (LCMS).

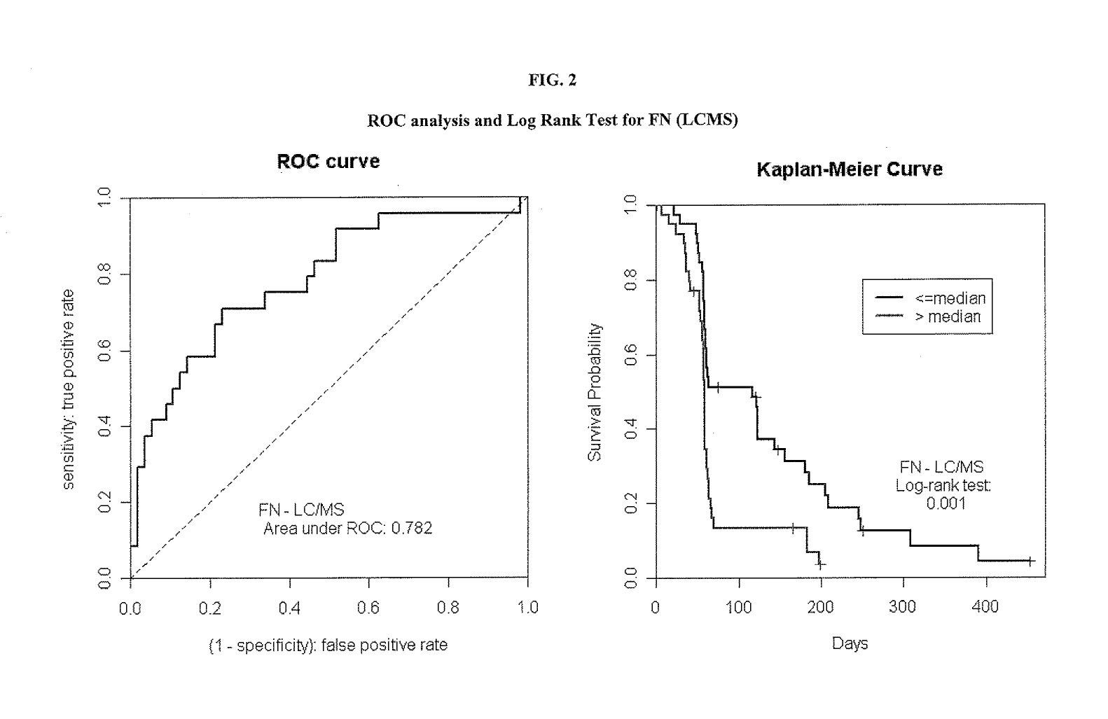

FIG. 3 provides box plots for the HPRG biomarker.

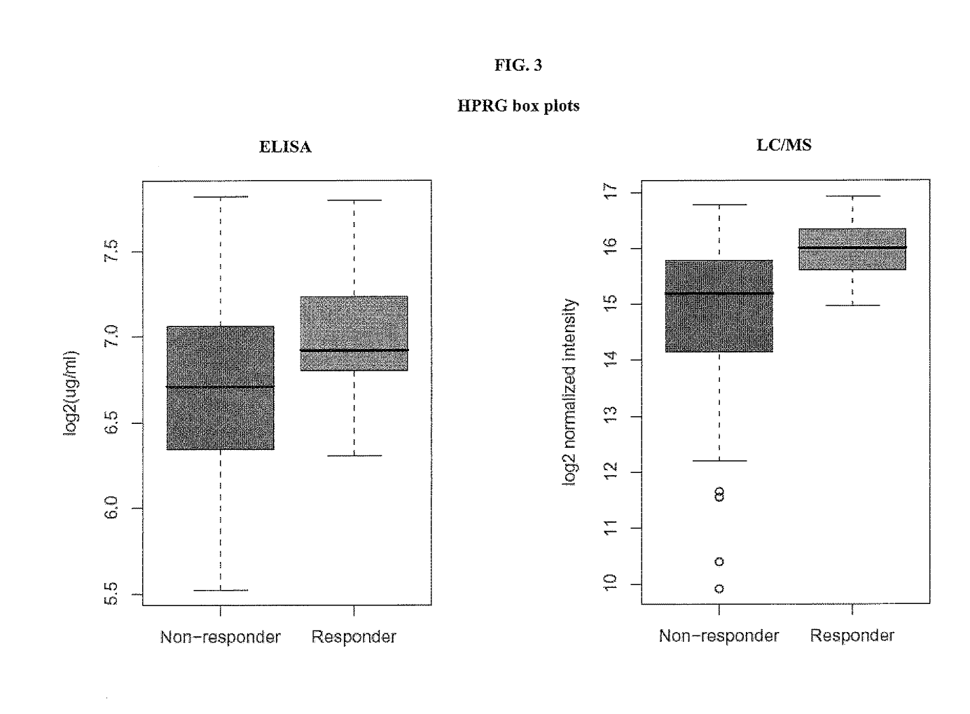

FIG. 4 provides an ROC analysis for the HPRG biomarker.

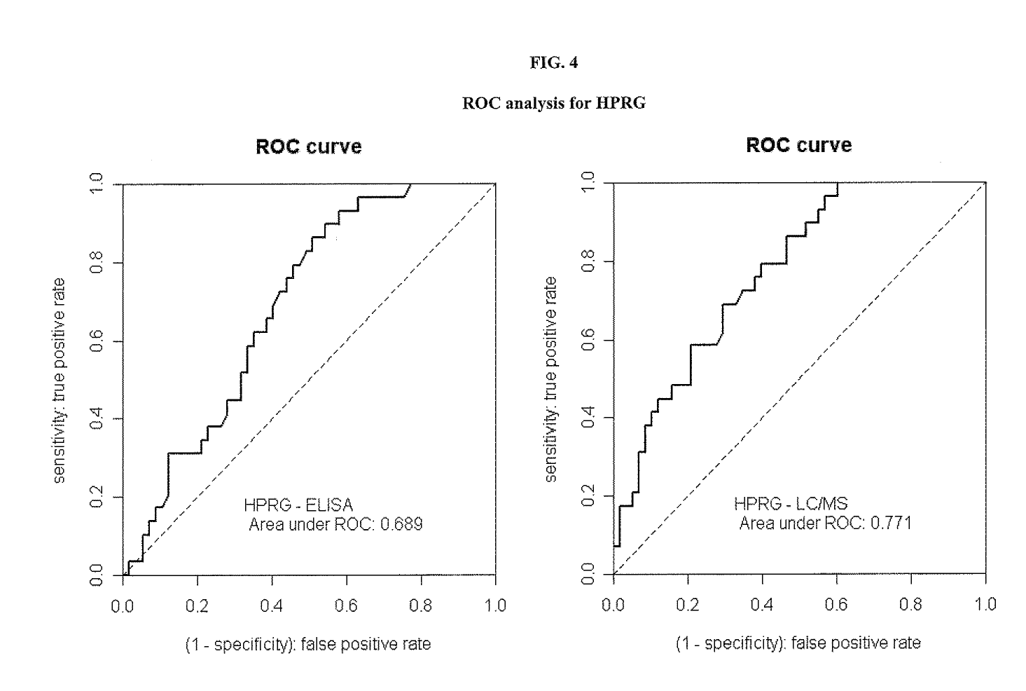

FIG. 5 provides Kaplan-Meier Curves for the HPRG biomarker.

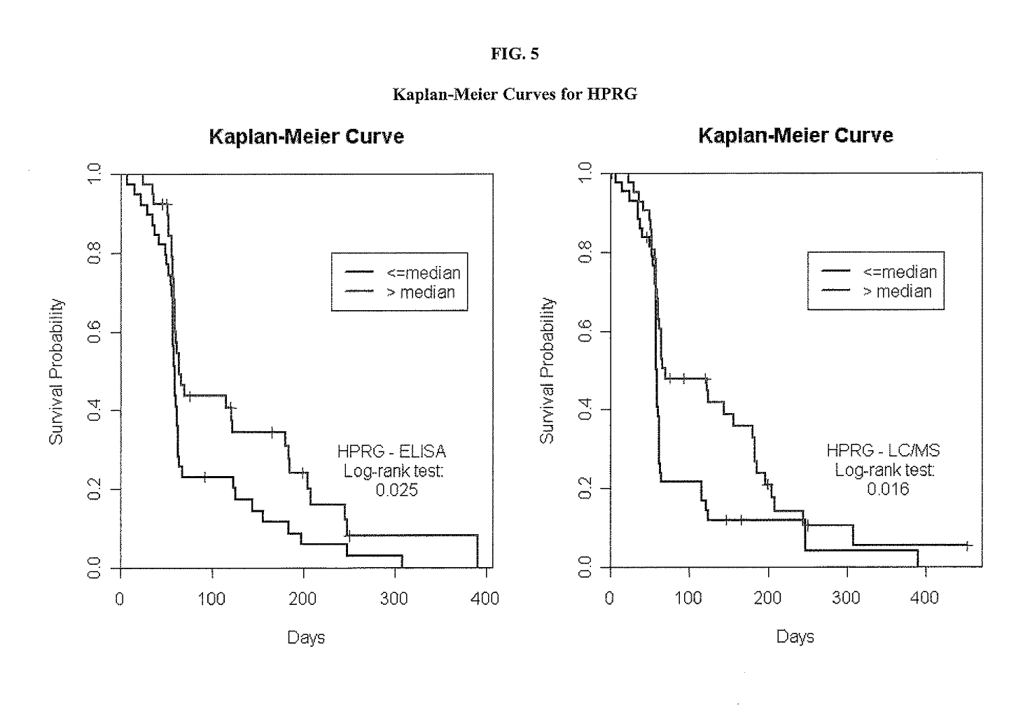

FIG. 6 provides box plots for the AHSG biomarker.

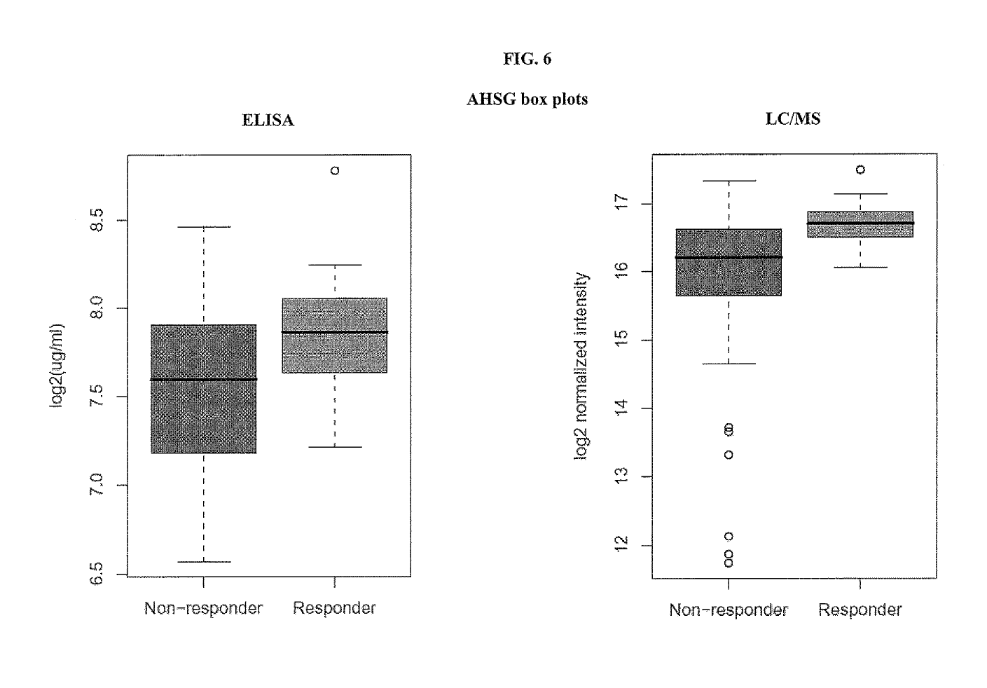

FIG. 7 provides an ROC analysis for the AHSG biomarker.

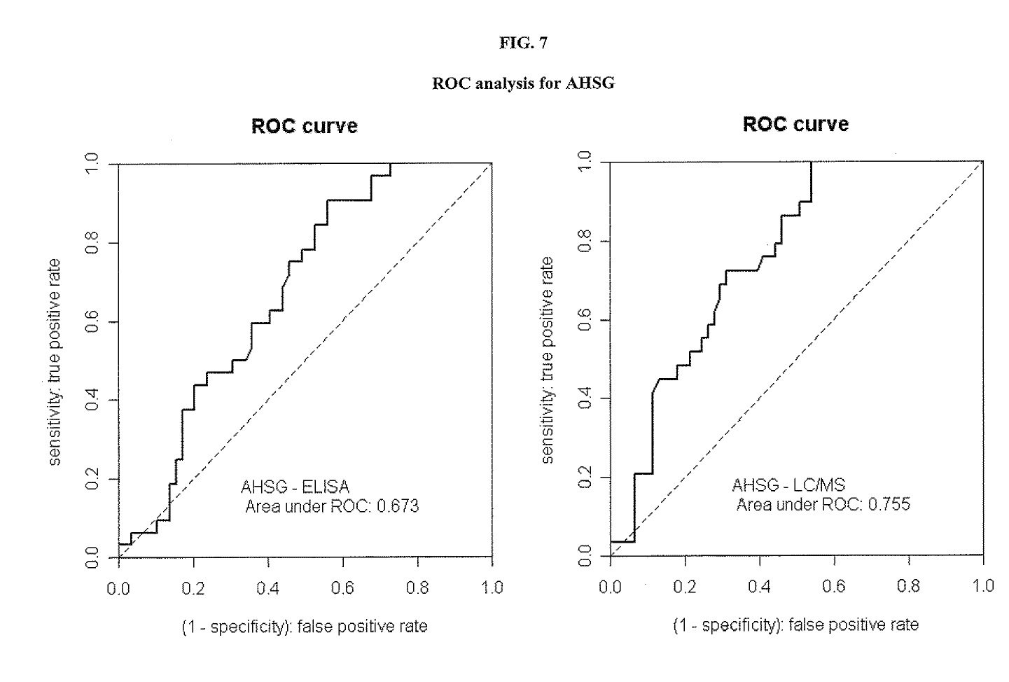

FIG. 8 provides Kaplan-Meier Curves for the AHSG biomarker.

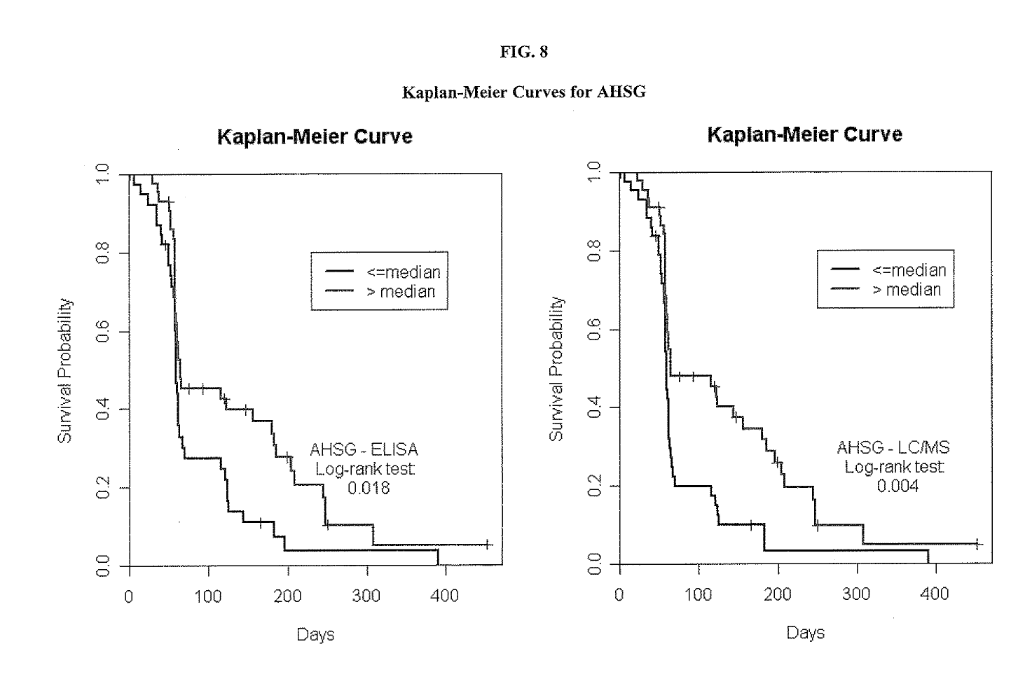

FIG. 9 provides a ROC schematic illustrating the utility of multi-peptide model (LCMS).

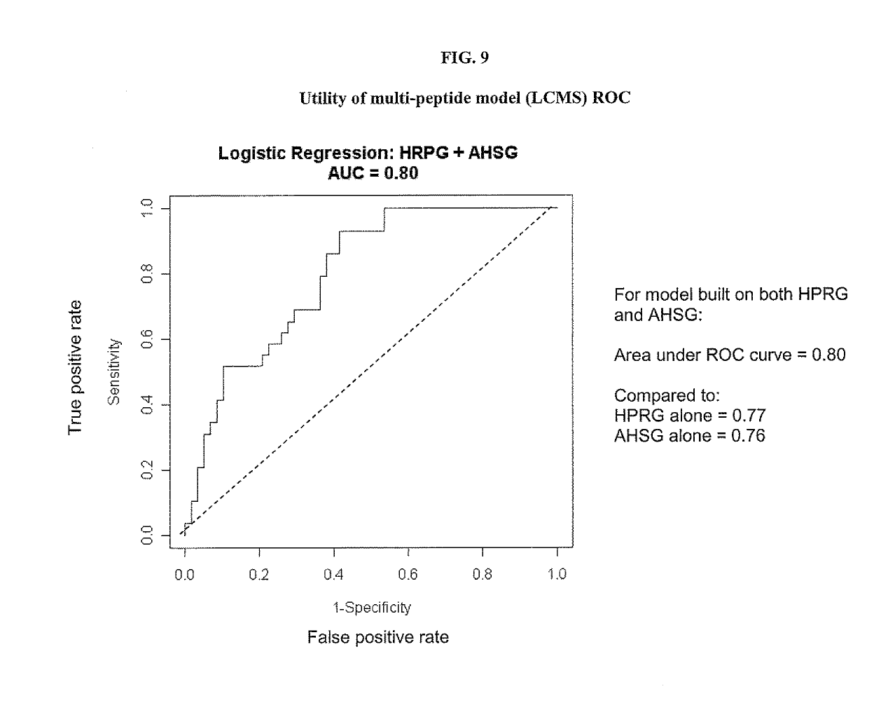

### DETAILED DESCRIPTION OF THE INVENTION

Identification of biomarkers that provide rapid and accessible readouts of efficacy, drug exposure, or clinical response is increasingly important in the clinical development of drug candidates. Embodiments of the invention include measuring changes in the levels of secreted proteins, or plasma biomarkers, which represent one category of biomarker. In one aspect, plasma samples, which represent a readily accessible source of material, serve as surrogate tissue for biomarker analysis.

In this study, soluble plasma biomarkers predictive of patient response to cetuximab, were investigated. The advantage of plasma biomarkers is that they are systemic, integrating information from primary and secondary tumors as well as the individual's response to their cancer. In addition, the measurement of plasma biomarkers is non-invasive, allowing multiple measurements to be taken over the course of treatment to assess changes.

LC/MS was performed on plasma samples taken from 90 patients enrolled in a Cetuximab monotherapy study for patients with refractory metastatic colorectal cancer. Plasma samples were taken prior to first dose. Candidate biomarkers were chosen based on differential expression between responders and non-responders and ability to predict time to progression. The top three biomarkers were selected for further evaluation by ELISA assay: Fibronectin (FN), Histidine Proline Rich Glycoprotein (HPRG), and Alpha-2-HS Glycoprotein (AHSG). It was hypothesized that these biomarkers play a role in cellular interactions with the extracellular matrix, and mediation of integrin-growth factor receptor interactions.

The invention provides biomarkers that respond to the modulation of a specific signal transduction pathway and also correlate with EGFR modulator sensitivity or resistance. These biomarkers can be employed for predicting response to one or more EGFR modulators. In one aspect, the biomarkers of the invention are those provided in Table 2, including both polynucleotide and polypeptide sequences. The invention also includes nucleotide sequences that hybridize to the polynucleotides provided in Table 2.

The biomarkers have expression levels in cells that may be dependent on the activity of the EGFR signal transduction pathway, and that are also highly correlated with EGFR modulator sensitivity exhibited by the cells. Biomarkers serve as useful molecular tools for predicting the likelihood of a response to EGFR modulators, preferably biological molecules, small molecules, and the like that affect EGFR kinase activity via direct or indirect inhibition or antagonism of EGFR kinase function or activity.

### Wild Type K-RAS and Mutated K-RAS

As used herein, wild type K-Ras can be selected from the K-Ras variant a and variant b nucleotide and amino acid sequences. Wild type K-Ras variant a has a nucleotide sequence that is 5436 nucleotides (GENBANK® Accession No. NM_{—}033360.2) and encodes a protein that is 189 amino acids (GENBANK® Accession No. NP_{—}203524.1). Wild type K-Ras variant b has a nucleotide sequence that is 5312 nucleotides (GENBANK® Accession No. NM_{—}004985.3) and encodes a protein that is 188 amino acids (GENBANK® Accession No. NP_{—}004976.2).

A mutated form of K-Ras is a nucleotide or amino acid sequence that differs from wild type K-Ras at least at one position, preferably at least one nucleotide position that encodes an amino acid that differs from wild type K-Ras. In one aspect, the mutated form of K-Ras includes at least one mutation in exon 1 and/or in exon 2. In another aspect, the mutated form of K-RAS includes at least one of the following mutations in exon 1 (base change (amino acid change)): 200G>A (V7M); 216G>C (G12A); 215G>T (G12C); 216G>A (G12D); 215G>C (G12R); 215G>A (G12S); 216G>T (G12V); 218G>T (G13C); 219G>A (G13D). In yet another respect, the mutated form of K-RAS includes at least one of the following mutations in exon 2 (base change (amino acid change)): CAA to CAT (Q61H).

Methods for detecting K-Ras mutations are well known in the art and include, for example, the methods described in PCT Publication No. WO 2005/118876.

### EGFR Modulators

As used herein, the term “EGFR modulator” is intended to mean a compound or drug that is a biological molecule or a small molecule that directly or indirectly modulates EGFR activity or the EGFR signal transduction pathway. Thus, compounds or drugs as used herein is intended to include both small molecules and biological molecules. Direct or indirect modulation includes activation or inhibition of EGFR activity or the EGFR signal transduction pathway. In one aspect, inhibition refers to inhibition of the binding of EGFR to an EGFR ligand such as, for example, EGF. In another aspect, inhibition refers to inhibition of the kinase activity of EGFR.

EGFR modulators include, for example, EGFR-specific ligands, small molecule EGFR inhibitors, and EGFR monoclonal antibodies. In one aspect, the EGFR modulator inhibits EGFR activity and/or inhibits the EGFR signal transduction pathway. In another aspect, the EGFR modulator is an EGFR monoclonal antibody that inhibits EGFR activity and/or inhibits the EGFR signal transduction pathway.

EGFR modulators include biological molecules or small molecules. Biological molecules include all lipids and polymers of monosaccharides, amino acids, and nucleotides having a molecular weight greater than 450. Thus, biological molecules include, for example, oligosaccharides and polysaccharides; oligopeptides, polypeptides, peptides, and proteins; and oligonucleotides and polynucleotides. Oligonucleotides and polynucleotides include, for example, DNA and RNA.

Biological molecules further include derivatives of any of the molecules described above. For example, derivatives of biological molecules include lipid and glycosylation derivatives of oligopeptides, polypeptides, peptides, and proteins.

Derivatives of biological molecules further include lipid derivatives of oligosaccharides and polysaccharides, e.g., lipopolysaccharides. Most typically, biological molecules are antibodies, or functional equivalents of antibodies. Functional equivalents of antibodies have binding characteristics comparable to those of antibodies, and inhibit the growth of cells that express EGFR. Such functional equivalents include, for example, chimerized, humanized, and single chain antibodies as well as fragments thereof.

Functional equivalents of antibodies also include polypeptides with amino acid sequences substantially the same as the amino acid sequence of the variable or hypervariable regions of the antibodies. An amino acid sequence that is substantially the same as another sequence, but that differs from the other sequence by means of one or more substitutions, additions, and/or deletions, is considered to be an equivalent sequence. Preferably, less than 50%, more preferably less than 25%, and still more preferably less than 10%, of the number of amino acid residues in a sequence are substituted for, added to, or deleted from the protein.

The functional equivalent of an antibody is preferably a chimerized or humanized antibody. A chimerized antibody comprises the variable region of a non-human antibody and the constant region of a human antibody. A humanized antibody comprises the hypervariable region (CDRs) of a non-human antibody. The variable region other than the hypervariable region, e.g., the framework variable region, and the constant region of a humanized antibody are those of a human antibody.

Suitable variable and hypervariable regions of non-human antibodies may be derived from antibodies produced by any non-human mammal in which monoclonal antibodies are made. Suitable examples of mammals other than humans include, for example, rabbits, rats, mice, horses, goats, or primates.

Functional equivalents further include fragments of antibodies that have binding characteristics that are the same as, or are comparable to, those of the whole antibody. Suitable fragments of the antibody include any fragment that comprises a sufficient portion of the hypervariable (i.e., complementarity determining) region to bind specifically, and with sufficient affinity, to EGFR tyrosine kinase to inhibit growth of cells that express such receptors.

Such fragments may, for example, contain one or both Fab fragments or the F(ab′)₂ fragment. Preferably, the antibody fragments contain all six complementarity determining regions of the whole antibody, although functional fragments containing fewer than all of such regions, such as three, four, or five CDRs, are also included.

In one aspect, the fragments are single chain antibodies, or Fv fragments. Single chain antibodies are polypeptides that comprise at least the variable region of the heavy chain of the antibody linked to the variable region of the light chain, with or without an interconnecting linker. Thus, Fv fragment comprises the entire antibody combining site. These chains may be produced in bacteria or in eukaryotic cells.

The antibodies and functional equivalents may be members of any class of immunoglobulins, such as IgG, IgM, IgA, IgD, or IgE, and the subclasses thereof.

In one aspect, the antibodies are members of the IgG1 subclass. The functional equivalents may also be equivalents of combinations of any of the above classes and subclasses.

In one aspect, EGFR antibodies can be selected from chimerized, humanized, fully human, and single chain antibodies derived from the murine antibody 225 described in U.S. Pat. No. 4,943,533.

In another aspect, the EGFR antibody is cetuximab (IMC-C225) which is a chimeric (human/mouse) IgG monoclonal antibody, also known under the tradename ERBITUX®. Cetuximab Fab contains the Fab fragment of cetuximab, i.e., the heavy and light chain variable region sequences of murine antibody M225 (U.S. Application No. 2004/0006212, incorporated herein by reference) with human IgG1 C_{H}1 heavy and kappa light chain constant domains. Cetuximab includes all three IgG1 heavy chain constant domains.

In another aspect, the EGFR antibody can be selected from the antibodies described in U.S. Pat. Nos. 6,235,883, 5,558,864 and 5,891,996. The EGFR antibody can be, for example, AGX-EGF (Amgen Inc.) (also known as panitumumab) which is a fully human IgG2 monoclonal antibody. The sequence and characterization of ABX-EGF, which was formerly known as clone E7.6.3, is disclosed in U.S. Pat. No. 6,235,883 at column 28, line 62 through column 29, line 36 and FIGS. 29-34, which is incorporated by reference herein. The EGFR antibody can also be, for example, EMD72000 (Merck KGaA), which is a humanized version of the murine EGFR antibody EMD 55900. The EGFR antibody can also be, for example: h-R3 (TheraCIM), which is a humanized EGFR monoclonal antibody; Y10 which is a murine monoclonal antibody raised against a murine homologue of the human EGFRvIII mutation; or MDX-447 (Medarex Inc.).

In addition to the biological molecules discussed above, the EGFR modulators useful in the invention may also be small molecules. Any molecule that is not a biological molecule is considered herein to be a small molecule. Some examples of small molecules include organic compounds, organometallic compounds, salts of organic and organometallic compounds, saccharides, amino acids, and nucleotides. Small molecules further include molecules that would otherwise be considered biological molecules, except their molecular weight is not greater than 450. Thus, small molecules may be lipids, oligosaccharides, oligopeptides, and oligonucleotides and their derivatives, having a molecular weight of 450 or less.

It is emphasized that small molecules can have any molecular weight. They are merely called small molecules because they typically have molecular weights less than 450. Small molecules include compounds that are found in nature as well as synthetic compounds. In one embodiment, the EGFR modulator is a small molecule that inhibits the growth of tumor cells that express EGFR. In another embodiment, the EGFR modulator is a small molecule that inhibits the growth of refractory tumor cells that express EGFR.

Numerous small molecules have been described as being useful to inhibit EGFR.

One example of a small molecule EGFR antagonist is IRESSA® (ZD1939), which is a quinozaline derivative that functions as an ATP-mimetic to inhibit EGFR. See, U.S. Pat. No. 5,616,582; WO 96/33980 at page 4. Another example of a small molecule EGFR antagonist is TARCEVA® (OSI-774), which is a 4-(substituted phenylamino)quinozaline derivative [6,7-Bis(2-methoxy-ethoxy)-quinazolin-4-yl]-(3-ethynyl-1-phenyl)amine hydrochloride] EGFR inhibitor. See WO 96/30347 (Pfizer Inc.) at, for example, page 2, line 12 through page 4, line 34 and page 19, lines 14-17. TARCEVA® may function by inhibiting phosphorylation of EGFR and its downstream PI3/Akt and MAP (mitogen activated protein) kinase signal transduction pathways resulting in p27-mediated cell-cycle arrest. See Hidalgo et al., Abstract 281 presented at the 37th Annual Meeting of ASCO, San Francisco, Calif., 12-15 May 2001.

Other small molecules are also reported to inhibit EGFR, many of which are thought to be specific to the tyrosine kinase domain of an EGFR. Some examples of such small molecule EGFR antagonists are described in WO 91/116051, WO 96/30347, WO 96/33980, WO 97/27199, WO 97/30034, WO 97/42187, WO 97/49688, WO 98/33798, WO 00/18761 and WO 00/31048. Examples of specific small molecule EGFR antagonists include C1-1033 (Pfizer Inc.), which is a quinozaline (N-[4-(3-chloro-4-fluoro-phenylamino)-7-(3-mprpholin-4-yl-propoxy)-quinazolin-6-yl]-acrylamide) inhibitor of tyrosine kinases, particularly EGFR and is described in WO 00/31048 at page 8, lines 22-6; PKI166 (Novartis), which is a pyrrolopyrimidine inhibitor of EGFR and is described in WO 97/27199 at pages 10-12; GW2016 (GlaxoSmithKline), which is an inhibitor of EGFR and HER2; EKB569 (Wyeth), which is reported to inhibit the growth of tumor cells that overexpress EGFR or HER2 in vitro and in vivo; AG-1478 (Tryphostin), which is a quinazoline small molecule that inhibits signaling from both EGFR and erbB-2; AG-1478 (Sugen), which is a bisubstrate inhibitor that also inhibits protein kinase CK2; PD 153035 (Parke-Davis) which is reported to inhibit EGFR kinase activity and tumor growth, induce apoptosis in cells in culture, and enhance the cytotoxicity of cytotoxic chemotherapeutic agents; SPM-924 (Schwarz Pharma), which is a tyrosine kinase inhibitor targeted for treatment of prostrate cancer; CP-546,989 (OSI Pharmaceuticals), which is reportedly an inhibitor of angiogenesis for treatment of solid tumors; ADL-681, which is a EGFR kinase inhibitor targeted for treatment of cancer; PD 158780, which is a pyridopyrimidine that is reported to inhibit the tumor growth rate of A4431 xenografts in mice; CP-358,774, which is a quinzoline that is reported to inhibit autophosphorylation in FINS xenografts in mice; ZD1839, which is a quinzoline that is reported to have antitumor activity in mouse xenograft models including vulvar, NSCLC, prostrate, ovarian, and colorectal cancers; CGP 59326A, which is a pyrrolopyrimidine that is reported to inhibit growth of EGFR-positive xenografts in mice; PD 165557 (Pfizer); CGP54211 and CGP53353 (Novartis), which are dianilnophthalimides. Naturally derived EGFR tyrosine kinase inhibitors include genistein, herbimycin A, quercetin, and erbstatin.

Further small molecules reported to inhibit EGFR and that are therefore within the scope of the present invention are tricyclic compounds such as the compounds described in U.S. Pat. No. 5,679,683; quinazoline derivatives such as the derivatives described in U.S. Pat. No. 5,616,582; and indole compounds such as the compounds described in U.S. Pat. No. 5,196,446.

Further small molecules reported to inhibit EGFR and that are therefore within the scope of the present invention are styryl substituted heteroaryl compounds such as the compounds described in U.S. Pat. No. 5,656,655. The heteroaryl group is a monocyclic ring with one or two heteroatoms, or a bicyclic ring with 1 to about 4 heteroatoms, the compound being optionally substituted or polysubstituted.

Further small molecules reported to inhibit EGFR and that are therefore within the scope of the present invention are bis mono and/or bicyclic aryl heteroaryl, carbocyclic, and heterocarbocyclic compounds described in U.S. Pat. No. 5,646,153.

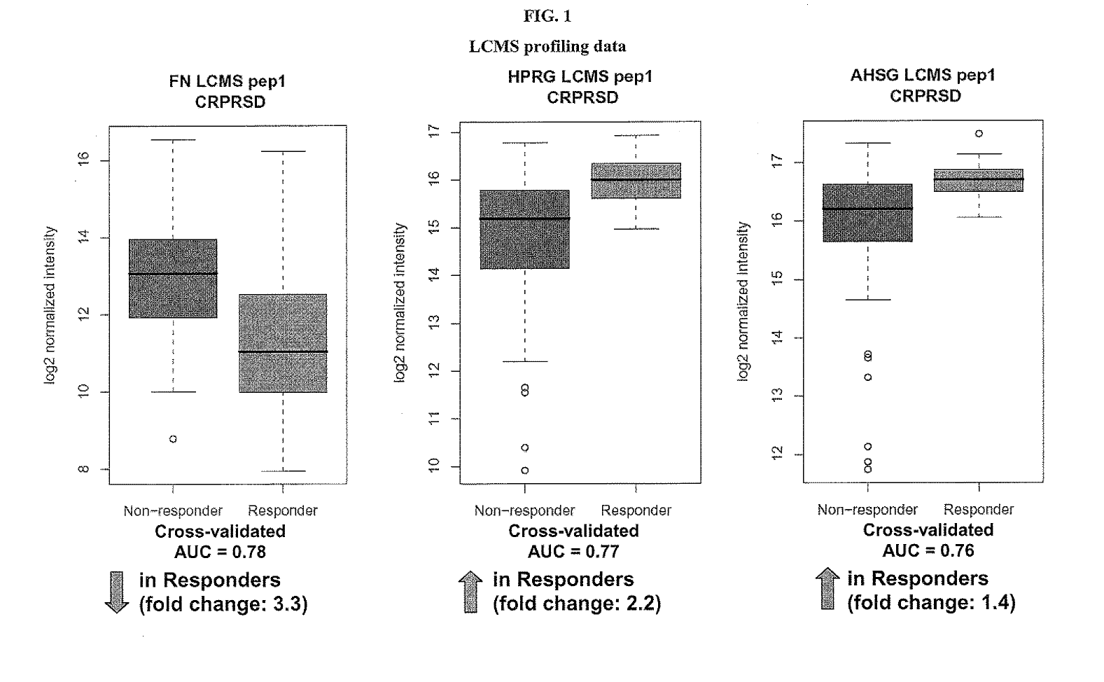

Further small molecules reported to inhibit EGFR and that are therefore within the scope of the present invention is the compound provided FIG. 1 of Fry et al., Science, 265:1093-1095 (1994) that inhibits EGFR. Further small molecules reported to inhibit EGFR and that are therefore within the scope of the present invention are tyrphostins that inhibit EGFR/HER1 and HER 2, particularly those in Tables I, II, III, and IV described in Osherov et al., J. Biol. Chem., 268(15):11134-11142 (1993).

Further small molecules reported to inhibit EGFR and that are therefore within the scope of the present invention is a compound identified as PD166285 that inhibits the EGFR, PDGFR, and FGFR families of receptors. PD166285 is identified as 6-(2,6-dichlorophenyl)-2-(4-(2-diethylaminoethyoxy)phenylamino)-8-methyl-8H-pyrido(2,3-d)pyrimidin-7-one having the structure shown in FIG. 1 on page 1436 of Panek et al., Journal of Pharmacology and Experimental Therapeutics, 283:1433-1444 (1997).

It should be appreciated that useful small molecule to be used in the invention are inhibitors of EGFR, but need not be completely specific for EGFR.

### Biomarkers and Biomarker Sets

The invention includes individual biomarkers and biomarker sets having both diagnostic and prognostic value in disease areas in which signaling through EGFR or the EGFR pathway is of importance, e.g., in cancers or tumors, in immunological disorders, conditions or dysfunctions, or in disease states in which cell signaling and/or cellular proliferation controls are abnormal or aberrant. The biomarker sets comprise a plurality of biomarkers such as, for example, a plurality of the biomarkers provided in Table 2, that highly correlate with resistance or sensitivity to one or more EGFR modulators.

The present invention encompasses the use of any one or more of the following as a biomarker for use in predicting EGFR-modulator response: Fibronectin; histidine-rich glycoprotein; alpha2-HS glycoprotein; Complement component 3; and/or ras suppressor protein 1.

The present invention also encompasses any combination of the aforementioned biomarkers, including, but not limited to: (i) Fibronectin; histidine-rich glycoprotein; alpha2-HS glycoprotein; Complement component 3; (ii) Fibronectin; histidine-rich glycoprotein; alpha2-HS glycoprotein; (iii) Fibronectin; histidine-rich glycoprotein; (iv) histidine-rich glycoprotein; alpha2-HS glycoprotein; Complement component 3; ras suppressor protein 1 (v) histidine-rich glycoprotein; alpha2-HS glycoprotein; Complement component 3; (v) histidine-rich glycoprotein; alpha2-HS glycoprotein; (vi) alpha2-HS glycoprotein; Complement component 3; ras suppressor protein 1; (vii) alpha2-HS glycoprotein; Complement component 3; (viii) Fibronectin, alpha2-HS glycoprotein; (ix) Fibronectin, Complement component 3; (x) Fibronect, ras suppressor protein 1; (xi) histidine-rich glycoprotein, Complement component 3; (xii) histidine-rich glycoprotein, ras suppressor protein 1; and (xiii) alpha2-HS glycoprotein, ras suppressor protein 1; in addition to any other combination thereof.

The biomarkers and biomarker sets of the invention enable one to predict or reasonably foretell the likely effect of one or more EGFR modulators in different biological systems or for cellular responses. The biomarkers and biomarker sets can be used in in vitro assays of EGFR modulator response by test cells to predict in vivo outcome. In accordance with the invention, the various biomarkers and biomarker sets described herein, or the combination of these biomarker sets with other biomarkers or markers, can be used, for example, to predict how patients with cancer might respond to therapeutic intervention with one or more EGFR modulators.

A biomarker and biomarker set of cellular gene expression patterns correlating with sensitivity or resistance of cells following exposure of the cells to one or more EGFR modulators provides a useful tool for screening one or more tumor samples before treatment with the EGFR modulator. The screening allows a prediction of cells of a tumor sample exposed to one or more EGFR modulators, based on the expression results of the biomarker and biomarker set, as to whether or not the tumor, and hence a patient harboring the tumor, will or will not respond to treatment with the EGFR modulator.

Measuring the level of expression of a biomarker and biomarker set provides a useful tool for screening one or more tumor samples before treatment of a patient with the EGFR-modulating agents. The screening allows a prediction of whether the cells of a tumor sample will respond favorably to the EGFR-modulating agents, based on the presence or absence of over-expression—such a prediction provides a reasoned assessment as to whether or not the tumor, and hence a patient harboring the tumor, will or will not respond to treatment with the EGFR-modulating agents.

A difference in the level of the biomarker that is sufficient to indicate whether the mammal will or will not respond therapeutically to the method of treating cancer can be readily determined by one of skill in the art using known techniques. The increase or decrease in the level of the biomarker can be correlated to determine whether the difference is sufficient to identify a mammal that will respond therapeutically. The difference in the level of the biomarker that is sufficient can, in one aspect, be predetermined prior to determining whether the mammal will respond therapeutically to the treatment. For example, the level of said biomarker may be established by identifying a standard, normal level of said biomarker in a mammal and using that level as a comparator to establish whether the test mammal has either an increased or decreased level of said marker. Preferably, the measured level is normalized relative to a reference, house keeping gene or protein, such as GADPH, actin, etc. In one aspect, the difference in the level of the biomarker is a difference in the mRNA level (measured, for example, by RT-PCR or a microarray), such as at least about a two-fold difference, at least about a three-fold difference, or at least about a four-fold difference in the level of expression, or more. In another aspect, the difference in the level of the biomarker is determined at the protein level by mass spectral methods or by FISH or by IHC. In another aspect, the difference in the level of the biomarker refers to a p-value of <0.05 in Anova analysis. In yet another aspect, the difference is determined in an ELISA assay.

The biomarker or biomarker set can also be used as described herein for monitoring the progress of disease treatment or therapy in those patients undergoing treatment for a disease involving an EGFR modulator.

The biomarkers also serve as targets for the development of therapies for disease treatment. Such targets may be particularly applicable to treatment of colorectal cancer. Indeed, because these biomarkers are differentially expressed in sensitive and resistant cells, their expression patterns are correlated with relative intrinsic sensitivity of cells to treatment with EGFR modulators. Accordingly, the biomarkers highly expressed in resistant cells may serve as targets for the development of new therapies for the tumors which are resistant to EGFR modulators, particularly EGFR inhibitors.

The level of biomarker protein and/or mRNA can be determined using methods well known to those skilled in the art. For example, quantification of protein can be carried out using methods such as ELISA, 2-dimensional SDS PAGE, Western blot, immunoprecipitation, immunohistochemistry, fluorescence activated cell sorting (FACS), or flow cytometry. Quantification of mRNA can be carried out using methods such as PCR, array hybridization, Northern blot, in-situ hybridization, dot-blot, TAQMAN®, or RNAse protection assay.

Identification of biomarkers that provide rapid and accessible readouts of efficacy, drug exposure, or clinical response is increasingly important in the clinical development of drug candidates. Embodiments of the invention include measuring changes in the levels of mRNA and/or protein in a sample to determine whether said sample contains increased expression of Fibronectin; histidine-rich glycoprotein; alpha2-HS glycoprotein; Complement component 3; and/or ras suppressor protein 1. In one aspect, said samples serve as surrogate tissue for biomarker analysis. These biomarkers can be employed for predicting and monitoring response to one or more EGFR-modulating agents. In one aspect, the biomarkers of the invention are one or more of the following: Fibronectin; histidine-rich glycoprotein; alpha2-HS glycoprotein; Complement component 3; and/or ras suppressor protein 1, including both polynucleotide and polypeptide sequences. In another aspect, the biomarkers of the invention are nucleotide sequences that, due to the degeneracy of the genetic code, encodes for a polypeptide sequence provided in the sequence listing.

The biomarkers serve as useful molecular tools for predicting and monitoring response to EGFR-modulating agents.

Methods of measuring the level of any given marker described herein may be performed using methods well known in the art, which include, but are not limited to PCR; RT-PCR; FISH; IHC; immuno-detection methods; immunoprecipitation; Western Blots; ELISA; radioimmunoassays; PET imaging; HPLC; surface plasmon resonance, and optical spectroscopy; and mass spectrometry, among others.

The biomarkers of the invention may be quantified using any immunospecific binding method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., Current Protocols in Molecular Biology, Vol. 1, John Wiley & Sons, Inc., New York (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% TRASYLOL®) supplemented with protein phosphatase and/or protease inhibitors (e.g., EDTA, PMSF, aprotinin, sodium vanadate), adding the antibody of interest (i.e., one directed to a biomarker of the present invention) to the cell lysate, incubating for a period of time (e.g., 1-4 hours) at 4° C., adding protein A and/or protein G SEPHAROSE® beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with SEPHAROSE® beads). For further discussion regarding immunoprecipitation protocols see, e.g., Ausubel et al., eds., Current Protocols in Molecular Biology, Vol. 1, John Wiley & Sons, Inc., New York at 10.16.1 (1994).

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds., Current Protocols in Molecular Biology, Vol. 1, John Wiley & Sons, Inc., New York at 10.8.1 (1994).

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds., Current Protocols in Molecular Biology, Vol. 1, John Wiley & Sons, Inc., New York at 11.2.1 (1994).

Alternatively, identifying the relative quantitation of the biomarker polypeptide(s) may be performed using tandem mass spectrometry; or single or multi dimensional high performance liquid chromatography coupled to tandem mass spectrometry. The method takes into account the fact that an increased number of fragments of an identified protein isolated using single or multi dimensional high performance liquid chromatography coupled to tandem mass spectrometry directly correlates with the level of the protein present in the sample. Such methods are well known to those skilled in the art and described in numerous publications, for example, Link, A. J., ed., 2-D Proteome Analysis Protocols, Humana Press (1999), ISBN: 0896035247; Chapman, J. R., ed., Mass Spectrometry of Proteins and Peptides, Humana Press (2000), ISBN: 089603609X.

As used herein the terms “modulate” or “modulates” or “modulators” refer to an increase or decrease in the amount, quality or effect of a particular activity, or the level of DNA, RNA, or protein detected in a sample.

### Microarrays

The invention also includes specialized microarrays, e.g., oligonucleotide microarrays or cDNA microarrays, comprising one or more biomarkers, showing expression profiles that correlate with either sensitivity or resistance to one or more EGFR modulators. Such microarrays can be employed in in vitro assays for assessing the expression level of the biomarkers in the test cells from tumor biopsies, and determining whether these test cells are likely to be resistant or sensitive to EGFR modulators. For example, a specialized microarray can be prepared using all the biomarkers, or subsets thereof, as described herein and shown in Table 2. Cells from a tissue or organ biopsy can be isolated and exposed to one or more of the EGFR modulators. In one aspect, following application of nucleic acids isolated from both untreated and treated cells to one or more of the specialized microarrays, the pattern of gene expression of the tested cells can be determined and compared with that of the biomarker pattern from the control panel of cells used to create the biomarker set on the microarray. Based upon the gene expression pattern results from the cells that underwent testing, it can be determined if the cells show a resistant or a sensitive profile of gene expression. Whether or not the tested cells from a tissue or organ biopsy will respond to one or more of the EGFR modulators and the course of treatment or therapy can then be determined or evaluated based on the information gleaned from the results of the specialized microarray analysis.

### Antibodies

The invention also includes antibodies, including polyclonal or monoclonal, directed against one or more of the polypeptide biomarkers. Such antibodies can be used in a variety of ways, for example, to purify, detect, and target the biomarkers of the invention, including both in vitro and in vivo diagnostic, detection, screening, and/or therapeutic methods.

### Kits

The invention also includes kits for determining or predicting whether a patient would be susceptible or resistant to a treatment that comprises one or more EGFR modulators. The patient may have a cancer or tumor such as, for example, colorectal cancer. Such kits would be useful in a clinical setting for use in testing a patient's biopsied tumor or other cancer samples, for example, to determine or predict if the patient's tumor or cancer will be resistant or sensitive to a given treatment or therapy with an EGFR modulator. The kit comprises a suitable container that comprises: one or more microarrays, e.g., oligonucleotide microarrays or cDNA microarrays, that comprise those biomarkers that correlate with resistance and sensitivity to EGFR modulators, particularly EGFR inhibitors; one or more EGFR modulators for use in testing cells from patient tissue specimens or patient samples; and instructions for use. In addition, kits contemplated by the invention can further include, for example, reagents or materials for monitoring the expression of biomarkers of the invention at the level of mRNA or protein, using other techniques and systems practiced in the art such as, for example, RT-PCR assays, which employ primers designed on the basis of one or more of the biomarkers described herein, immunoassays, such as enzyme linked immunosorbent assays (ELISAs), immunoblotting, e.g., Western blots, or in situ hybridization, and the like.

### Application of Biomarkers and Biomarker Sets

The biomarkers and biomarker sets may be used in different applications. Biomarker sets can be built from any combination of biomarkers listed in Table 2 to make predictions about the effect of an EGFR modulator in different biological systems. The various biomarkers and biomarkers sets described herein can be used, for example, as diagnostic or prognostic indicators in disease management, to predict how patients with cancer might respond to therapeutic intervention with compounds that modulate the EGFR, and to predict how patients might respond to therapeutic intervention that modulates signaling through the entire EGFR regulatory pathway.

The biomarkers have both diagnostic and prognostic value in diseases areas in which signaling through EGFR or the EGFR pathway is of importance, e.g., in immunology, or in cancers or tumors in which cell signaling and/or proliferation controls have gone awry.

In one aspect, cells from a patient tissue sample, e.g., a tumor or cancer biopsy, can be assayed to determine the expression pattern of one or more biomarkers prior to treatment with one or more EGFR modulators. In one aspect, the tumor or cancer is colorectal. Success or failure of a treatment can be determined based on the biomarker expression pattern of the cells from the test tissue (test cells), e.g., tumor or cancer biopsy, as being relatively similar or different from the expression pattern of a control set of the one or more biomarkers. Thus, if the test cells show a biomarker expression profile which corresponds to that of the biomarkers in the control panel of cells which are sensitive to the EGFR modulator, it is highly likely or predicted that the individual's cancer or tumor will respond favorably to treatment with the EGFR modulator. By contrast, if the test cells show a biomarker expression pattern corresponding to that of the biomarkers of the control panel of cells which are resistant to the EGFR modulator, it is highly likely or predicted that the individual's cancer or tumor will not respond to treatment with the EGFR modulator.

The invention also provides a method of monitoring the treatment of a patient having a disease treatable by one or more EGFR modulators. The isolated test cells from the patient's tissue sample, e.g., a tumor biopsy or tumor sample, can be assayed to determine the expression pattern of one or more biomarkers before and after exposure to an EGFR modulator wherein, preferably, the EGFR modulator is an EGFR inhibitor. The resulting biomarker expression profile of the test cells before and after treatment is compared with that of one or more biomarkers as described and shown herein to be highly expressed in the control panel of cells that are either resistant or sensitive to an EGFR modulator. Thus, if a patient's response is sensitive to treatment by an EGFR modulator, based on correlation of the expression profile of the one or biomarkers, the patient's treatment prognosis can be qualified as favorable and treatment can continue. Also, if, after treatment with an EGFR modulator, the test cells don't show a change in the biomarker expression profile corresponding to the control panel of cells that are sensitive to the EGFR modulator, it can serve as an indicator that the current treatment should be modified, changed, or even discontinued. This monitoring process can indicate success or failure of a patient's treatment with an EGFR modulator and such monitoring processes can be repeated as necessary or desired.

The methods of the present invention may be performed, at least in part, on any machine or apparatus capable of identifying, measuring, normalizing, and/or quantifying the expression levels of the biomarkers of the present invention. Such machines preferably include any necessary programming, logic, and/or instructions needed to carry out the identification, measurement, normalization, and/or quantification of the biomarkers of the present invention. Examples of such machines include, but are not limited to, PCR machines, ELISA machines, mass spectrometers, IHC machines, HPLC machines, proteomic machines, western blot machines, FACS machines, etc.

In addition, the methods of the present invention necessarily constitute the transformation of physiological information (e.g., biomarker identity, biomarker quantification, and/or biomarker expression level determination of any biomarker disclosed herein, and/or the presence or absence of a biomarker such as, but not limited to, K-RAS mutations, etc.) into clinically relevant information a physician or health care provider may reasonably rely upon to make informed, treatment decisions.

In order to facilitate a further understanding of the invention, the following examples are presented primarily for the purpose of illustrating more specific details thereof. The scope of the invention should not be deemed limited by the examples, but to encompass the entire subject matter defined by the claims.

### REFERENCES

1. Laura Moro, Laura Dolce, Sara Cabodi, Elena Bergatto, Elisabetta Boeri Erba, Monica Smeriglio, Emilia Turco, Saverio Francesco Retta, Maria Gabriella Giuffrida, Mascia Venturino, Jasminka Godovac-Zimmermann, Amedeo Conti, Erik Schaefer, Laura Beguinot, Carlo Tacchetti, Paolo Gaggini, Lorenzo Silengo, Guido Tarone, and Paola Defilippi. Integrin-induced Epidermal Growth Factor (EGF) Receptor Activation Requires c-Src and p130Cas and Leads to Phosphorylation of Specific EGF Receptor Tyrosines. J Biol Chem Vol. 277, Issue 11, 9405-9414 2002.
2. K M Yamada and S Even-Ram, Integrin regulation of growth factor receptors. Nat Cell Biol 2002 4(4):E75-6
3. Paolo M Comoglio, Carla Boccaccio and Livio Trusolino, Interactions between growth factor receptors and adhesion molecules: breaking the rules, Curr Opin Cell Biol Volume 15, Issue 5, October 2003, Pages 565-571.
4. W Guo and F G Giancotti, Integrin signaling during tumor progression, Nat Rev Mol Cell Biol W 2004 October; 5(10): 816-26.
5. B A Hocevar, T L Brown, P H Howe, TGF-β induces fibronectin synthesis through a c-Jun N-terminal kinase-dependent, Smad4-independent pathway. EMBO J 1999, 18,1345-1356.
6. Johan Dixelius, Anna-Karin Olsson, Asa Thulin, Chunsik Lee, Irja Johansson and Lena Claesson-Welsh. Minimal Active Domain and Mechanism of Action of the Angiogenesis Inhibitor Histidine-Rich Glycoprotein, Cancer Research, 2006, 66, 2089-2097.
7. Demetriou M, Binkert C, Sukhu B, Tenenbaum H C, Dennis J W., Fetuin/alpha2-HS glycoprotein is a transforming growth factor-beta type II receptor mimic and cytokine antagonist., J Biol Chem 1996 271(22):12755-61.
8. Swallow C J, Partridge E A, Macmillan J C, Tajirian T, DiGuglielmo G M, Hay K, Szweras M, Jahnen-Dechent W, Wrana J L, Redston M, Gallinger S, Dennis J W., Alpha2HS-glycoprotein, an antagonist of transforming growth factor beta in vivo, inhibits intestinal tumor progression. Cancer Research, 2004.
9. Kwak J Y, Ma T Z, Yoo M J, Choi B H, Kim H G, Kim S R, Yim C Y, Kwak Y G., The comparative analysis of serum proteomes for the discovery of biomarkers for acute myeloid leukemia., Exp Hematol 2004 32(9):836-42.

### EXAMPLE 1

### method of Identifying Plasma Biomarkers Predictive of Patient Response to EGFR Modulation

### Methods

### Patient Treatment and Clinical End Points

One hundred and ten patients with metastatic CRC were enrolled onto a cetuximab monotherapy study. Patients were eligible if they had histologically documented metastatic CRC. Patients must have received at least one prior chemotherapeutic regimen for advanced disease or have refused prior treatment. A standard cetuximab dosing regimen (400 mg/m² loading dose, followed by 250 mg/m² weekly) was followed for the first 3 weeks of therapy; thereafter, patients were eligible for dose escalation every 3 weeks to a maximum dose of 400 mg/m² provided that they had not experienced more than grade 2 skin rash. Median duration of study therapy was 9 weeks. All patients underwent a pretreatment blood draw for plasma collection. Tumor response was evaluated every 9 weeks (one cycle of therapy) according to the modified WHO criteria. (Khambata-Ford, 2007)

### Protein Profiling and Data Analysis

Plasma Depletion and Trypsin Digestion. Ninety samples were received for protein profiling. Samples were randomized prior to processing. For each sample, 300 μl of plasma was used for protein profiling. The Agilent high capacity multiple affinity removal system (MARS, 4.6×100 mm affinity column, Agilent) was utilized to remove six high-abundant proteins from plasma. This technology enables higher plasma loads for the removal of albumin, IgG, antitrypsin, IgA, transferrin and haptoglobin in a single step. MARS depletion was carried out in accordance with manufacturer instructions. (Agilent, Delaware). A standard tryptic digest was applied to all samples (details described elsewhere).

Solid Phase Extraction (SPE). Samples were split into two equal volumes and transferred to two 96 well plates in a randomized fashion. These plates were subjected to solid phase extraction to desalt using a C18 SPE plate (3M) with a vacuum manifold. An 8-tip liquid handler (MultiPROBE II HT Expanded System, Perkin Elmer) was used for sample handling. Washing buffer consisted of 0.1% TFA and the elution buffer consisted of 90% acetonitrile in 0.1% TFA (Ho, 2004).

Duplication and Lyophilization. Twenty microliters of eluate per sample was withheld for peptide concentration determination. The remainder of each sample was then split across two separate 96-well plates (VWR). Of the duplicate plates, one was used for LC/MS profiling, the other for LC/MS/MS target identification. A SPEEDVAC® unit was used to lyophilize the samples to dryness (Thermo Savant, Holbrook, N.Y.). Samples were stored at −80° C. until LC/MS was performed (Ho, 2004).

LC/MS. Samples of tryptic peptides were separated on an Agilent ZORBAX® 3005B-C18 column (0.5×150 mm, 3.5 μm) equipped with a 0.5 μm pre-column filter (Opti-solve). The buffers were delivered at a flow rate of 12 μL/min on an Agilent 1100 Capillary HPLC system. The two mobile phases used were as follows: buffer A: water+0.2% isopropyl alcohol, 0.1% acetic acid and 0.001% trifluoroacetic acid; buffer B: 95% acetonitrile+0.2% isopropyl alcohol, 0.1% acetic acid and 0.001% trifluoroacetic acid. An optimized nonlinear gradient was used to separate the peptides (shown below).

| | Time (min) | | | | | | | |
|---|---|---|---|---|---|---|---|---|
| | 0 | 2 | 4 | 64 | 69 | 71 | 71.1 | 80 |
| % B | 0 | 0 | 10 | 40 | 100 | 100 | 0 | Stop |

Samples were re-dissolved in reconstitution solution in volume according to the peptide concentration to ensure that all samples had the same final concentration prior to injection onto the LC/MS system. The reconstitution solution contained three internal standards [20] at 0.5 μg each /mL in 0.2% isopropyl alcohol, 5% acetic acid and 0.001% trifluoroacetic acid. These internal standards were used to monitor chromatographic reproducibility and MS sensitivity. Eight microliters of sample was injected for each run using an Agilent 1100 micro well plate sampler equipped with an 8 μL loop and chilled at 4° C. To achieve optimum mass accuracy, a lock mass solution, Glu-Fibrinopeptide B (GFP, Sigma, 1 mg/L in 50% Buffer B), was introduced through a Valco-type mixing tee into the flowing system immediately after the RPLC column outlet. The delivery of the lock mass solution was performed using a separate Agilent 1100 isocratic pump at 1 μL/min. The analysis was performed on a Qtof Ultima operated in electrospray positive ionization mode with “V” optics configuration. Mass spectra were acquired for the mass range from 300 to 1800 Da. Each acquisition was 80 minutes long, with a 1 second scan time and a 0.1 second inter-scan delay.

### Data Analysis

Data deconvolution, retention time adjustment, quantile normalization, data clustering, and error modeling were performed on raw LC/MS spectra using a suite of in-house software. LC/MS intensities, accurate mass and retention time were obtained for approximately 10,000 unique peptide ions. This peptide ion list formed the basis of all subsequent statistical analyses.

For each peptide ion, the inventors performed the unpooled variance T-test (Satterwaite's test), logistic regression and Cox proportional hazard test. For the T-test and logistic regression, patients were classified as either responders or non-responders. Responders consisted of patients labeled as Complete Responder (CR), Partial Responders (PR), Stable Disease (SD). Non-responders consisted of patients labeled as Progressive Disease (PD). These designations were based on established guidelines from the World Health Organization (WHO) (World Health Organization. Handbook for Reporting Results of Cancer Treatment, Offset Publication 48, Geneva, Switzerland: World Health Organization; 1979) and the Response Evaluation Criteria in Solid Tumors (Therasse, P. et al., “New guidelines to evaluate the response to treatment in solid tumors”, European Organization for Research and Treatment of Cancer, National Cancer Institute of the United States, National Cancer Institute of Canada, J. Natl. Cancer Inst., 92:205-216 (2000)). Peptides which passed each of these three tests with p-value<0.01 were selected for cross-validation. Based on these criteria, 52 candidates were selected.

Cross-validation was performed by randomly partitioning subjects into 4 subsets of roughly the same size stratified by patient response. One subset comprises the test set, while the others pooled together make up the training set. The same three statistical tests were performed on the training set, and again a peptide was selected if P<0.01 for each test. Logistic regression was performed on the test set for the peptides and the AUC, the area under the receiver operator curve (ROC), was computed. This process was repeated such that each partition served as the held out testing set. This entire process was iterated 200 times, for 200 different random partitions of the data. The AUC values were averaged to give one cross validated AUC. Six peptide candidates with a cross validated AUC value greater than or equal to 0.75 were selected for further analysis.

### Target Identification

LC/MS/MS. The list of peptide ions with statistical significance generated from our data analysis was subjected to MS/MS analysis. Samples were rerun on the same LC/MS platform in data-dependant mode in which the MS survey scan switches to MS/MS product scan when targeted peptide ions were found at the same retention time and mass. MS/MS spectra were generated and submitted to a SEQUEST® search, which yielded protein identifications.

Viper. Identification of peptide ions can also be achieved by running the program Viper. Viper matches peptide ions to a peptide database with accurate mass and retention time information to yield putative protein identifications. (Patwardhan, 2006).

### Enzyme-Linked Immunosorbent Assay (ELISA)

Alpha 2 HS glycoprotein (AHSG) in human plasma was measured using ELISA kits provided by BioVendor. (Candler, N.C.). Plates pre-coated with capture polyclonal anti-human AHSG, reference standards, quality controls (QC), HRP conjugated detection antibody (Conjugate Solution), Substrate Solution and Stop Solution were all included in the kits. Plates were incubated with 100 μL of plasma samples, reference standards or QCs for 1 hour at RT, shaken at 300 rpm on an orbital microplate shaker. Plates were washed, and 100 μL of Conjugate Solution was added per well for 1 hour at RT while samples were again shaken at 300 rpm. After washing, 100 μL per well of Substrate Solution was added for 10 minutes at RT, followed by adding 100 μL per well of Stop Solution. The absorbance at 450 nm was measured using a SPECTRAMAX® plate reader (Molecular Devices Inc, Sunnyvale, Calif.). The concentration of AHSG was determined using a calibration curve based on the reference standards.

HPRG in human plasma was measured using ELISAs built in house. All antibodies, reference standards, streptavidin-horseradish peroxidase (HRP), the HRP substrate and stop solution were obtained from R&D Systems Inc. (Minneapolis, Minn.). Ninety-six—well flat bottom plates were coated with mouse anti-human HPRG capture antibody (4 μg/mL) in phosphate-buffered saline overnight at 4° C. Plates were washed and blocked for 10 minutes in 200 μL per well of SUPERBLOCK® (Pierce, Rockford, Ill.) and then washed and incubated with 50 μL of either plasma samples or reference standards for 1 hour at RT. Plates were washed, and 100 μL of biotin anti-human HPRG was added per well for 1 hour at RT. After washing, 100 μL per well of streptavidin-HRP was added for 20 minutes at RT, followed by washing and incubation with 100 μL per well of substrate solution at RT. The reaction was stopped by adding 50 μL per well of stop solution, and the absorbance at 450 nm was measured using a SPECTRAMAX® plate reader (Molecular Devices Inc., Sunnyvale, Calif.). The concentration of HPRG was determined using a calibration curve based on the reference standards.

### Results

A total of 90 patients' plasma samples were used for the plasma profiling experiment. Of these 90 patients, 29 were classified as responder (CR+PR+SD), and 61 as non-responder (PD). (CR=“complete response/remission”; PR=“partial response/remission”; SD=“stable disease”; PD=“progressive disease”). The response rate for this study was 32.2% (Table 1). LC/MS peptide profiling was performed on each sample. After raw spectra were processed the inventors performed statistical analysis on the resulting 10,000 putative peptide ions.

Our initial analysis resulted in 52 candidate peptide ions which were selected for cross-validation. Our top candidate peptides were those whose cross-validated AUC was greater than or equal to 0.75 (see Methods). This yielded a list of six peptides for further analysis. These unique peptides were subsequently sequenced using tandem mass spectrometry or the VIPER program to identify the proteins from which the peptides originated. Our list of six unique peptides generated four protein identifications: Fibronectin (FN), Histidine-Proline Rich Glycoprotein (HPRG), α2-HS-Glycoprotein (AHSG), and Complement Component 3 (C3) (Table 2).

Fibronectin is present in higher concentrations in the plasma of Non-Responders with an AUC of 0.78. Conversely, HPRG and AHSG are both present at higher concentrations in the Responder population, with AUCs of 0.77 and 0.76, respectively (FIG. 1). Accordingly, increased expression levels of histidine-rich glycoprotein; alpha2-HS glycoprotein; Complement component 3; and/or ras suppressor protein 1 relative to a standard level of at least one of these biomarkers indicates an increased likelihood a mammal will respond therapeutically to an anti-EGFR therapy for treating cancer. Conversely, decreased expression levels of fibronectin relative to a standard level indicates an increased likelihood a mammal will respond therapeutically to an anti-EGFR therapy for treating cancer.

To confirm the identification of these proteins, ELISA assays were developed for Fibronectin, HPRG, and AHSG and run using excess sample from the same clinical trial. C3 was omitted from further study due to the presence of peptides from multiple splice isoforms whose intensities were not correlated, and because as part of the immune response, it is unlikely to be a signal with specificity for colorectal cancer.

### Discussion

In this study the inventors have identified three potential plasma biomarkers predictive of patient response to Cetuximab therapy. Advantages of plasma biomarkers such as these are that
1. They are systemic measurements, not specific to one given tumor (primary or secondary), thus they can integrate information from the entire body.
2. Measurements of plasma biomarkers are non-invasive, allowing repeated measurements to follow changes in the course of a disease.
   Each of the biomarkers the inventors focus on here play a role in extracellular signaling and specifically in mediating interactions between integrins and growth factor receptors, including EGFR.

Fibronectin is a part of the extracellular matrix, which is degraded and remodeled during cancer cell invasion. This process is mediated by the response of integrins to cancer signaling pathways. Fibronectin can influence cell signaling through interactions with several classes of integrins. First, Fibronectin binding to the α5β1 integrin can lead to ligand-independent activation of the EGFR (Moro et al., J. Biol. Chem. (2002); Yamada et al., Nat. Cell Biol. (2002); Comoglio et al., Curr. Opin. Cell Biol. (2003)). Second, Fibronectin binding to αvβ3 integrins can enhance the signaling of several growth factor signaling pathways (ERBB2, PDGFR, VEGFR) through cooperative interactions between the integrins and growth factor receptors (Guo et al., Nat. Rev. Mol. Cell. Bio. (2004); Comoglio et al., Curr. Opin. Cell Biol. (2003)). Finally the activation of αvβ6 integrins on Fibronectin binding may mediate a positive feedback cycle. Fibronectin binding to αvβ6 integrins leads to the activation of TGFβ. Reciprocally, TGFβ leads to increased Fibronectic expression (Guo et al., Nat. Rev. Mol. Cell Bio. (2004); Hoceval et al., EMBO J. (1999)). In each of these scenarios an increase in Fibronectin would be expected to decrease the response to Cetuximab, as the inventors have seen in our study.

Like Fibronectin, HPRG exerts its influence by affecting integrin/growth factor receptor interactions. HPRG is thought to disrupt integrin crosstalk with VEGFR. By preventing VEGFR from phosphorylating its FAK substrate, HPRG inhibits angiogenesis (Dix Dixelius et al., Cancer Research (2006)). The inventors would expect patients with increased HPRG to have less activation of the VEGFR pathway, and thus have a more positive response profile than patients with lower HPRG levels. This is what the inventors have found in our study.

AHSG is an antagonist of TGFβ (Demetriou et al., J. Biol. Chem. (1996)). TGFβ is known to be involved in the epithelial-mesenchymal transition. In addition to inhibiting this process, AHSG may also inhibit Fibronectin synthesis through TGFβ activation. Based on these roles for AHSG and TGFβ, the inventors would expect a decrease in AHSG to lead to a decreased response to Cetuximab, which is what the inventors have found in our study. In human colorectal cancer tumor samples it has been shown that AHSG levels are 3-fold lower in tumor tissue than in normal tissue (Swallow et al., Cancer Research (2004)). In leukemia patients AHSG levels were found to be decreased in serum (Kwak et al., Exp. Hematol. (2004)).

The cancer signaling pathway initiated by a specific growth factor receptor is a complex system. However it does not act in isolation responding to the presence of absence of its ligand or ligands. Rather, its activity can be influenced through other mechanisms as well. This is exemplified by the numerous ways activated integrins can influence growth factor receptor signaling, resulting in ligand-independent activation, collaborative signaling, or establishing a positive feedback cycle between integrin and growth factor receptor activation (Moro et al., J. Biol. Chem. (2002); Yamada et al., Nat. Cell Biol. (2002); Comoglio et al., Curr. Opin. Cell Biol. (2003); Guo et al., Nat. Rev. Mol. Cell Bio. (2004)). Interestingly, each of our top three plasma biomarkers play a role in these processes.

### EXAMPLE 2

### Method of Assess Expression Profile of Biomarkers Using MRNA from Tissue and Cell Source

Total RNA may be purified using RNEASY® system (Qiagen, CA, USA). Mixed Oligo-d(T)₁₅ primers may be used to generate single-stranded cDNAs using the SUPERSCRIPT® First-strand Synthesis kit (Invitrogen, CA, USA). Levels for each gene of interest and GAPDH transcripts may be analyzed using an Applied Biosystems 7900HT Sequence Detection System. Mixed primer/probe sets for each transcript of interest (for example, ELISA assay: Fibronectin (FN), catalog # Hs00415006_m1; histidine-rich glycoprotein, catalog # Hs00426275_m1; alpha2-HS glycoprotein, catalog # Hs00155659_m1; Complement component 3, catalog # Hs00355887_g1; and/or ras suppressor protein 1, catalog # Hs00541590_s1) may be obtained from Applied Biosystems and used according to the manufacturer's instructions.

Expression levels of transcripts of interest may then be normalized to endogenous GAPDH transcripts. Comparisons may be made between samples by ΔΔCt comparative analysis using manufacturer's software (Applied Biosystems). Briefly, ΔCT=(MDR CT)-(GAPDH CT); ΔΔCT=(ΔCT^{Probe1}-ΔCT^{Probe2}); and Fold change=2^{ΔΔC}.

### EXAMPLE 3

### Production of Antibodies Against the Biomarkers

Antibodies against the biomarkers can be prepared by a variety of methods. For example, cells expressing a biomarker polypeptide can be administered to an animal to induce the production of sera containing polyclonal antibodies directed to the expressed polypeptides. In one aspect, the biomarker protein is prepared and isolated or otherwise purified to render it substantially free of natural contaminants, using techniques commonly practiced in the art. Such a preparation is then introduced into an animal in order to produce polyclonal antisera of greater specific activity for the expressed and isolated polypeptide.

In one aspect, the antibodies of the invention are monoclonal antibodies (or protein binding fragments thereof). Cells expressing the biomarker polypeptide can be cultured in any suitable tissue culture medium, however, it is preferable to culture cells in Earle's modified Eagle's medium supplemented to contain 10% fetal bovine serum (inactivated at about 56° C.), and supplemented to contain about 10 g/l nonessential amino acids, about 1,00 U/ml penicillin, and about 100 μg/ml streptomycin.

The splenocytes of immunized (and boosted) mice can be extracted and fused with a suitable myeloma cell line. Any suitable myeloma cell line can be employed in accordance with the invention, however, it is preferable to employ the parent myeloma cell line (SP2/0), available from the ATCC® (Manassas, Va.). After fusion, the resulting hybridoma cells are selectively maintained in HAT medium, and then cloned by limiting dilution as described by Wands et al. (Gastroenterology, 80:225-232 (1981)). The hybridoma cells obtained through such a selection are then assayed to identify those cell clones that secrete antibodies capable of binding to the polypeptide immunogen, or a portion thereof.

Alternatively, additional antibodies capable of binding to the biomarker polypeptide can be produced in a two-step procedure using anti-idiotypic antibodies. Such a method makes use of the fact that antibodies are themselves antigens and, therefore, it is possible to obtain an antibody that binds to a second antibody. In accordance with this method, protein specific antibodies can be used to immunize an animal, preferably a mouse. The splenocytes of such an immunized animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce the formation of further protein-specific antibodies.

### EXAMPLE 4

### Immunofluorescence Assays

The following immunofluorescence protocol may be used, for example, to verify EGFR biomarker protein expression on cells or, for example, to check for the presence of one or more antibodies that bind EGFR biomarkers expressed on the surface of cells. Briefly, LAB-TEK® II chamber slides are coated overnight at 4° C. with 10 micrograms/milliliter (μg/ml) of bovine collagen Type II in DPBS containing calcium and magnesium (DPBS++). The slides are then washed twice with cold DPBS++ and seeded with 8000 CHO-CCR5 or CHO pC4 transfected cells in a total volume of 125 μl and incubated at 37° C. in the presence of 95% oxygen/5% carbon dioxide.

The culture medium is gently removed by aspiration and the adherent cells are washed twice with DPBS++ at ambient temperature. The slides are blocked with DPBS++ containing 0.2% BSA (blocker) at 0-4° C. for one hour. The blocking solution is gently removed by aspiration, and 125 μl of antibody containing solution (an antibody containing solution may be, for example, a hybridoma culture supernatant which is usually used undiluted, or serum/plasma which is usually diluted, e.g., a dilution of about 1/100 dilution). The slides are incubated for 1 hour at 0-4° C. Antibody solutions are then gently removed by aspiration and the cells are washed five times with 400 μl of ice cold blocking solution. Next, 125 μl of 1 μg/ml rhodamine labeled secondary antibody (e.g., anti-human IgG) in blocker solution is added to the cells. Again, cells are incubated for 1 hour at 0-4° C.

The secondary antibody solution is then gently removed by aspiration and the cells are washed three times with 400 μl of ice cold blocking solution, and five times with cold DPBS++. The cells are then fixed with 125 μl of 3.7% formaldehyde in DPBS++ for 15 minutes at ambient temperature. Thereafter, the cells are washed five times with 400 μl of DPBS++ at ambient temperature. Finally, the cells are mounted in 50% aqueous glycerol and viewed in a fluorescence microscope using rhodamine filters. The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

The entire disclosure of each document cited (including patents, patent applications, journal articles, abstracts, laboratory manuals, books, GENBANK® Accession numbers, SWISS-PROT® Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

TABLE 1
Definition of Responders and Non-Responders

| | | # of | # of | % |
|---|---|---|---|---|
| | Response | Responder | Non-Responder | Response |
| 1 | CR + PR | 7 | 83 | 7.8% |
| 2 | CR + PR + SD27WKS | 11 | 79 | 12.2% |
| 3 | CR + PR + SD18WKS | 18 | 72 | 20.0% |
| 4 | CR + PR + SD | 29 | 61 | 32.2% |

TABLE 2
Top Plasma Marker Candidates

| | | | | | Four-fold cross validation | | |
|---|---|---|---|---|---|---|---|
| | | P-value | | | 200 iterations | | |
| | | | Logistic | Cox Prop. | # times | | |
| Protein ID | Refseq # | T-test | Regression | Hazard | selected | Avg. Order | Avg. AUC |
| Fibronectin | NP_997647, NP_997643, | 2.5e(−4) | 5.0e(−5) | 3.1e(−4) | 200 | 5.1 | 0.78 |
| | NP_997641, NP_997639, | | | | | | |
| | NP_002017, NP_997640 | | | | | | |
| histidine-rich glycoprotein | NP_000403 | 1.6e(−6) | 3.2e(−6) | 2.0e(−3) | 200 | 5.7 | 0.77 |
| alpha2-HS glycoprotein | NP_001613 | 3.2e(−6) | 1.6e(−5) | 1.3e(−3) | 200 | 7.4 | 0.76 |
| Complement component 3 | NP_000055 | 2.5e(−5) | 3.2e(−5) | 3.2e(−3) | 200 | 10.9 | 0.75 |
| ras suppressor protein 1 | NP_036557, NP_689937 | 2.0e(−3) | 2.5e(−4) | 6.3e(−4) | 200 | 25.8 | 0.74 |

What is claimed is:

1. A method for predicting the likelihood a mammal will respond therapeutically to an EGFR modulator comprising the step of measuring the level of at least one biomarker in a biological sample of said mammal selected from the group consisting of: Fibronectin; histidine-rich glycoprotein; alpha2-HS glycoprotein; Complement component 3; and/or ras suppressor protein 1 in plasma; wherein an increase or decrease in the level of the at least one biomarker relative to a standard level indicates an increased or decreased likelihood the mammal will respond therapeutically to said EGFR modulator in treating cancer or other proliferative condition.

2. The method of claim 1 wherein said at least one biomarker further comprises one or more of the following additional biomarker(s): epiregulin and amphiregulin.

3. The method of claim 1 wherein said at least one biomarker further comprises at least one additional biomarker selected from Table 2.

4. The method of claim 1 wherein said measuring step comprises use of one or more of the methods selected from the group consisting of: PCR-based methods; RT-PCR; immunohistochemistry (IHC); HPLC; PET imaging; mass-spectrometry (LC-MS, LC-MS/MS MALDI-MS); FISH; ELISA; SDS PAGE; 2-dimensional SDS PAGE, Western blot, immunoprecipitation, fluorescence activated cell sorting (FACS), flow cytometry; radioimmunoassays; and surface plasmon resonance.

5. The method of claim 1 that further comprises the step of determining whether said cancer cells have the presence of a mutated K-RAS, wherein detection of a mutated K-RAS indicates a decreased likelihood the mammal will respond therapeutically to said method of treating cancer.

6. A method for predicting the likelihood a mammal will respond therapeutically to a method of treating cancer comprising administering an EGFR modulator, wherein the method comprises:
(a) measuring in the mammal the level of at least one biomarker that comprises Fibronectin; histidine-rich glycoprotein; alpha2-HS glycoprotein;
Complement component 3; and/or ras suppressor protein 1;
(b) administering an EGFR modulator to said mammal; and
(c) following the administering step (b), measuring in said biological sample the level of the at least one biomarker,
wherein an increase or decrease in the level of the at least one biomarker measured in step (c) compared to the level of the at least one biomarker measured in step (a) indicates an increased or decreased likelihood the mammal will respond therapeutically to said method of treating cancer.

7. The method of claim 6 wherein said at least one biomarker further comprises one or more additional biomarker(s) selected from Table 2.

8. The method of claim 7 wherein said measuring step comprises use of one or more of the methods selected from the group consisting of: PCR-based methods; RT-PCR; immunohistochemistry (IHC); HPLC; PET imaging; mass-spectrometry (LC-MS, LC-MS/MS MALDI-MS); FISH; ELISA; SDS PAGE; 2-dimensional SDS PAGE, Western blot, immunoprecipitation, fluorescence activated cell sorting (FACS), flow cytometry; radioimmunoassays; and surface plasmon resonance.

9. The method of claim 9 that further comprises the step of determining whether said cancer cells have the presence of a mutated K-RAS, wherein detection of a mutated K-RAS indicates a decreased likelihood that that the mammal will respond therapeutically to said method of treating cancer.

10. The method according to claim 1 or 6, wherein said mammal is human.

11. The method according to any one of claim 1 or 6, wherein said mammal is selected from the group consisting of: rat, mouse, dog, rabbit, pig sheep, cow, horse, cat, primate, and monkey.

12. The method according to claim 1 or 6, wherein said EGFR modulator is an anti-EGFR antibody or a small molecule EGFR inhibitor.

13. The method according to claim 12, wherein said anti-EGFR antibody is monoclonal, polyclonal or single chain antibodies.

14. The method according to claim 13, wherein said anti-EGFR antibody is a fully human, humanized, or chimeric antibody.

15. The method according to claim 1 or 6, wherein said EGFR modulator is cetuximab or panitumumab.
